(19) United States
(12) Patent Application Publication
(10) Pub. No.: US 2018/0321224 A1
(43) Pub. Date: Nov. 8, 2018

(54) COMPOSITIONS AND METHODS FOR INCREASING HEPATOCYTE FUNCTIONAL LIFETIME IN VITRO

(71) Applicant: Colorado State University Research Foundation, Fort Collins, CO (US)

(72) Inventors: Salman R. Khetani, Chicago, IL (US); Matthew D. Davidson, Philadelphia, PA (US)

(73) Assignee: Colorado State University Research Foundation, Fort Collins, CO (US)

(21) Appl. No.: 15/775,301

(22) PCT Filed: Nov. 11, 2016
(86) PCT No.: PCT/US16/61638

§ 371 Date: May 10, 2018

Related U.S. Application Data
(60) Provisional application No. 62/253,964, filed on Nov. 11, 2015.

(51) Int. Cl. G01N 33/50; C12N 5/071

(52) U.S. Cl. CPC G01N 33/5067; C12N 5/067; C12N 2502/13; C12N 2501/33; C12N 2500/34; G01N 33/5014

(57) ABSTRACT

The present disclosure provides a culture medium formulation comprising human serum that can enhance functions of primary human hepatocytes, improve morphology, promote bile canaliculi formation and extend hepatocyte functional lifetime in vitro for over 10 weeks as compared to ˜3-4 weeks when using a conventional culture medium containing serum from bovine sources. The provided long-term culture model can be used to screen drugs for their efficacious and/or toxic effects over several weeks, improve drug-transporter assays via the larger bile canaliculi network, and to model several chronic liver diseases such as hepatitis, type 2 diabetes, malaria, liver fibrosis, liver cancer, and fatty liver disease.

### CROSS REFERENCE TO RELATED APPLICATIONS

This application claims the benefit of U.S. Provisional Application No. 62/253,964, filed Nov. 11, 2015, the disclosure of which is hereby incorporated by reference in its entirety.

### GOVERNMENTAL RIGHTS

This invention was made with government support under CBET-1351909 awarded by the National Science Foundation and 1R03EB019184-01 awarded by the National Institutes of Health. The government has certain rights in the invention.

### FIELD OF THE INVENTION

The present disclosure relates to cell culture media and in particular to an improved cell culture medium formulation for in vitro co-cultures of primary human hepatocytes with a non-parenchymal cell population, and related methods of use.

### BACKGROUND OF THE INVENTION

Primary human hepatocytes (PHHs), the main cell type of the human liver, rapidly lose their phenotypic functions under conventional culture formats that rely exclusively on extracellular matrices (ECM). Co-culture of PHHs with stromal cells from both within and outside the liver in either 2-dimensional or 3-dimensional configurations can prolong hepatic phenotype to ˜4 weeks in vitro. The lifetime of a hepatocyte in vivo is around 150 days; however, no current culture platform can keep hepatocyte highly functional for that length of time. The culture medium plays an important role in hepatocyte survival in any culture configuration; however, most models utilize culture media that have supra-physiologic levels of insulin and contain serum from animal sources (i.e. bovine). There is a need in the art for improving the culture media formulation to further prolong the lifetime of PHHs in vitro. Such a medium can result in hepatocyte cultures that have applications in testing the effects of chronically administered drugs and diseases that affect the liver, such as hepatitis, type 2 diabetes, malaria, liver fibrosis, liver cancer and fatty liver disease.

### SUMMARY OF THE INVENTION

In an aspect, the disclosure provides a method of culturing a population of human hepatocytes in vitro, comprising co-culturing the population of human hepatocytes with at least one non-human, non-parenchymal cell population wherein the composition is incubated with a culture medium comprising human serum obtained from at least one donor. The culture medium can comprise from about 5% to about 10% vol/vol human serum. The culture medium further can comprise about 0.1 to about 1 nM insulin. Specifically, the culture medium can comprise about 0.5 nM insulin. The culture medium further can comprise about 1 to about 25 mM glucose. Specifically, the culture medium can comprise about 5 mM glucose. The population of hepatocytes and the at least one non-human, non-parenchymal cell population can be maintained in vitro for at least 6 weeks. The population of hepatocytes and the at least one non-human, non-parenchymal cell population can be maintained in vitro for at least 8 weeks. The population of hepatocytes and the at least one non-human, non-parenchymal cell population can be maintained in vitro for at least 10 weeks. The human hepatocytes can be primary human hepatocytes. The human serum can be obtained from at least one human donor suffering from a disorder of the liver. The disorder of the liver is selected from type-2 diabetes, metabolic syndrome, non-alcoholic fatty liver disease (NAFLD), non-alcoholic steatohepatitis (NASH), and cardiovascular disease. At least one of the non-human, non-parenchymal cell populations can comprise non-human stromal cells. The stromal cells can be selected from the group consisting of fibroblasts, fibroblast-derived cells, macrophages, endothelial cells, stellate cells, pericytes, inflammatory cells, cholangiocytes and other types of stromal cells, and combinations thereof. The stromal cells can comprise murine embryonic fibroblasts. The murine embryonic fibroblasts can comprise 3T3-J2 murine embryonic fibroblasts.

In another aspect, the disclosure provides a composition comprising a population of human hepatocytes and at least one non-human, non-parenchymal cell population in co-culture in vitro, wherein the composition is incubated with a culture medium comprising human serum for at least 1 hour. The culture medium can comprise from about 5% to about 10% vol/vol human serum. The culture medium further can comprise about 0.1 to about 1 nM insulin. Specifically, the culture medium can comprise about 0.5 nM insulin. The culture medium further can comprise about 1 to about 25 mM glucose. Specifically, the culture medium can comprise about 5 mM glucose. The culture medium can comprise about 0.1 to about 1 nM insulin and about 1 to about 25 mM glucose. The composition can be incubated with the culture medium for at least 24 hours. Additionally, the composition can be incubated with the culture medium for at least 7 days. The population of hepatocytes and the at least one non-human, non-parenchymal cell population can be maintained in vitro for at least 6 weeks. The population of hepatocytes and the at least one non-human, non-parenchymal cell population can be maintained in vitro for at least 8 weeks. The population of hepatocytes and the at least one non-human, non-parenchymal cell population can be maintained in vitro for at least 10 weeks. The human hepatocytes can be primary human hepatocytes. The human serum can be obtained from at least one human donor suffering from a disorder of the liver. The disorder of the liver is selected from type-2 diabetes, metabolic syndrome, non-alcoholic fatty liver disease (NAFLD), non-alcoholic steatohepatitis (NASH), and cardiovascular disease. At least one of the non-human, non-parenchymal cell populations can comprise non-human stromal cells. The stromal cells can be selected from the group consisting of fibroblasts, fibroblast-derived cells, macrophages, endothelial cells, stellate cells, pericytes, inflammatory cells, cholangiocytes and other types of stromal cells, and combinations thereof. The stromal cells can comprise murine embryonic fibroblasts. The murine embryonic fibroblasts can comprise 3T3-J2 murine embryonic fibroblasts.

In still another aspect, the disclosure provides a method of identifying a candidate test compound for use in treating a disorder of the liver, the method comprising: contacting a composition of the disclosure with the test compound; maintaining the composition for a time and under conditions sufficient to allow an effect of the test compound on the hepatocytes; measuring at least one indicator of hepatic function in the hepatocytes to obtain a test measurement, or applying hepatocyte imaging technology (HIAT) to the hepatocytes to obtain a test image; and comparing the test measurement to a control measurement from the hepatocytes before contact with the test compound, or the test image to a control image of the hepatocytes before contact with the test compound, wherein a difference between the test and control is indicative of whether the test compound is a candidate for use in treating a disorder of the liver. The at least one indicator of hepatic function can be selected from the group consisting of albumin production, urea production, ATP production, glutathione production, enzyme activity, lipid accumulation, liver gene expression, and liver protein expression in the hepatocytes. The at least one inducible liver enzyme can be selected from CYP2C9 (luciferin-H), CYP3A4 (luciferin-IPA), a combination of CYP1A1, CYP1A2, CYP2B6, CPY2A6, and CYP2D6 (luciferin ME-EGE), and any combination thereof.

In still yet another aspect, the disclosure provides a method of determining the toxicity of a test compound, the method comprising: contacting a composition of the disclosure with the test compound; maintaining the composition for a time and under conditions sufficient to allow an effect of the test compound on the hepatocytes; measuring at least one indicator of hepatic function in the hepatocytes to obtain a test measurement, or applying hepatocyte imaging technology (HIAT) to the hepatocytes to obtain a test image; and comparing the test measurement to a control measurement from the hepatocytes before contact with the test compound, or the test image to a control image of the hepatocytes before contact with the test compound, wherein a difference between the test and control is indicative of hepatotoxicity of the test compound. The at least one indicator of hepatic function can be selected from the group consisting of albumin production, urea production, ATP production, glutathione production, enzyme activity, lipid accumulation, liver gene expression, and liver protein expression in the hepatocytes. The at least one inducible liver enzyme can be selected from CYP2C9 (luciferin-H), CYP3A4 (luciferin-IPA), a combination of CYP1A1, CYP1A2, CYP2B6, CPY2A6, and CYP2D6 (luciferin ME-EGE), and any combination thereof.

In a different aspect, the disclosure provides a method of identifying drug metabolites, the method comprising: contacting a composition of the disclosure with a drug; maintaining the composition for a time and under conditions sufficient to allow the generation of metabolites; and identifying the metabolites. In an aspect, the method comprises: contacting a composition of the disclosure with a drug; maintaining the composition for a time and under conditions sufficient to allow the generation of metabolites; and isolating the hepatocytes for the purpose of metabolite identification. In another aspect, the method comprises: contacting a composition of the disclosure with a drug; maintaining the composition for a time and under conditions sufficient to allow the generation of metabolites; and isolating the supernatant for the purpose of metabolite identification. In still another aspect, the method comprises: contacting a composition of the disclosure with a drug; maintaining the composition for a time and under conditions sufficient to allow the generation of metabolites; and storing the composition for future metabolite identification. In another aspect, the method comprises: identifying the metabolites in a supernatant isolated from compositions of the disclosure that have been contacted with a drug.

In other aspect, the disclosure provides a method of predicting hepatic clearance of a drug, the method comprising: contacting a composition of the disclosure with the drug; maintaining the composition for a time and under conditions sufficient to allow an effect of the drug on the hepatocytes; measuring the drug concentration in the composition; and determining a hepatic clearance value from the drug concentration measurement. In an aspect, the method comprises: contacting a composition of the disclosure with the drug; maintaining the composition for a time and under conditions sufficient to allow an effect of the drug on the hepatocytes; and isolating the hepatocytes for the purpose of determining a hepatic clearance value from a drug concentration measurement. In another aspect, the method comprises: contacting a composition of the disclosure with the drug; maintaining the composition for a time and under conditions sufficient to allow an effect of the drug on the hepatocytes; and isolating the supernatant for the purpose of determining a hepatic clearance value from a drug concentration measurement. In still another aspect, the method comprises: contacting a composition of the disclosure with the drug; maintaining the composition for a time and under conditions sufficient to allow an effect of the drug on the hepatocytes; and storing the composition for future determination of a hepatic clearance value. In another aspect, the method comprises: a determining hepatic clearance value from a drug concentration measurement obtained from compositions of the disclosure that have been contacted with a drug.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A, FIG. 1B, FIG. 1C, FIG. 1D, FIG. 1E, FIG. 1F, FIG. 1G, FIG. 1H and FIG. 1I depict a schematic and images showing that physiologically relevant medium prolongs the lifetime of hepatocytes in micropatterned co-cultures. (FIG. 1A) MPCC fabrication scheme. Representative phase contrast images of MPCC hepatocyte islands in traditional medium (FIG. 1B, FIG. 1C, FIG. 1D, FIG. 1E) and physiologic medium (FIG. 1F, FIG. 1G, FIG. 1H, FIG. 1I) over time. Scale bar represents 400 μm.

FIG. 2A, FIG. 2B, FIG. 2C and FIG. 2D depict graphs showing that hepatocyte functional lifetime is significantly longer in physiologic medium. MPCCs were carried out in traditional or physiologic medium for 8-10 weeks and assessed for CYP3A4 (FIG. 2A), and CYP2A6 (FIG. 2B) enzyme activity. Urea synthesis (FIG. 2C) and albumin production (FIG. 2D) were assessed in cultures treated with traditional or physiologic medium over 10 weeks and at 3 weeks of culture, respectively. Error bars represent SD.

FIG. 3A, FIG. 3B, FIG. 3C, FIG. 3D, FIG. 3E and FIG. 3F depict images showing that polarized hepatocyte transporters remain intact in physiologic medium. MPCCs were cultured for 4 weeks in physiologic (FIG. 3A, FIG. 3B, FIG. 3C) or traditional (FIG. 3D, FIG. 3E, FIG. 3F) medium and then assessed for bile canaliculi function, with CDCFDA transporter dye, and corresponding phase contrast imaging.

FIG. 4A, FIG. 4B and FIG. 4C depict a diagram and graphs showing that hepatocyte insulin sensitivity is retained in physiologically relevant medium. (FIG. 4A) Diagram describing how to calculate insulin resistance in MPCCs. Insulin resistance after 2 and 4 weeks of culture in traditional (FIG. 4B) and physiologic (FIG. 4C) medium. Error bars represent SD.

FIG. 5A, FIG. 5B, FIG. 5C, FIG. 5D, FIG. 5E, FIG. 5F, FIG. 5G, FIG. 5H, FIG. 5I, FIG. 5J, FIG. 5K, FIG. 5L and FIG. 5M depict graphs showing that physiologically relevant medium allows for sensitive and specific hepatotoxicity screening as well as drug-drug interactions. MPCCs were cultured in physiologic medium for 10 days and then placed in a liver toxicity screen (FIG. 5A-FIG. 5J) or assessed for induction capabilities (FIG. 5K, FIG. 5L, FIG. 5M). (FIG. 5A-FIG. 5E) Urea synthesis in treatments with liver toxins after the first and second drug treatment or day 3 and 5 of dosing. (FIG. 5A) Diclofenac, (FIG. 5B) Troglitazone, (FIG. 5C) Clozapine, (FIG. 5D) Amiodarone, and (FIG. 5E) Piroxicam. (FIG. 5F-FIG. 5J) Urea synthesis in treatments with non-toxins after the first and second drug treatment or day 3 and 5 of dosing. (FIG. 5F) Aspirin, (FIG. 5G) Rosiglitazone, (FIG. 5H) Dexamethasone, (FIG. 5I) Miconazole, and (FIG. 5J) Prednisone. CYP3A4 (FIG. 5K), CYP1A2 (FIG. 5L) and CYP2C9 (FIG. 5M) enzyme induction with prototypical inducers. Error bars represent SD.

FIG. 6A, FIG. 6B, FIG. 6C and FIG. 6D depict graphs showing optimization of culture medium to enable proper comparison of physiologically relevant medium to traditional medium. (FIG. 6A) Urea synthesis in MPCCs cultured in medium containing bovine serum and physiologic or the normal high insulin. (FIG. 6B) Urea synthesis in MPCCs cultured in medium containing human serum and physiologic or the normal high insulin. (FIG. 6C, FIG. 6D) 3 week relative CYP3A4 activity in MPCCs cultured in medium containing bovine serum (FIG. 6C) or human serum (FIG. 6D) and physiologic or the normal high insulin, normalized to the high insulin control. Error bars represent SD.

FIG. 7A, FIG. 7B, FIG. 7C and FIG. 7D depict graphs showing optimization of serum concentration in cell culture medium. MPCCs were cultured in medium with different percentages of serum for 2 weeks and albumin and urea production were assessed. (FIG. 7A, FIG. 7B) Albumin production in cultures with bovine (FIG. 7A) and human (FIG. 7B) serum at different percentages. (FIG. 7C, FIG. 7D) Urea synthesis in cultures with bovine (FIG. 7C) and human (FIG. 7D) serum at different percentages. Error bars represent SD.

FIG. 8 depicts a graph showing insulin resistance overtime with varying insulin concentration and serum type. MPCCs were cultured in traditional medium, with the normal high insulin concentration or physiologic insulin concentration (low insulin), or physiologic medium and assessed for insulin resistance as described in FIG. 4. This figure shows the same data depicted in FIG. 4 for comparison. Error bars represent SD.

FIG. 9A and FIG. 9B depict graphs showing CYP1A2 and CYP2C9 activity in MPCCs cultured in traditional and physiologic medium. MPCCs were cultured in traditional or physiologic medium for 3 weeks and assessed for CYP1A2 (FIG. 9A) and CYP2C9 (FIG. 9B) activity. Error bars represent SD.

FIG. 10A, FIG. 10B, FIG. 10C, FIG. 10D and FIG. 10E depict MPCCs in bovine serum (BS) or human serum (HS) containing culture medium. HS improves retention of bile canaliculi formation (FIG. 10A, FIG. 10C, 4 weeks), prototypical hepatic morphology (FIG. 10B, FIG. 10D, 10 weeks), and CYP3A4 enzyme activity (FIG. 10E). “1” and “2” in FIG. 10E refer to different lots of sera.

### DETAILED DESCRIPTION OF THE INVENTION

The present disclosure is based in part on the surprising discovery by the inventors that murine non-parenchymal cells survive and grow in a culture medium containing human serum. Accordingly, the present disclosure provides a culture medium formulation comprising human serum that can enhance functions of primary human hepatocytes in co-culture with a population of non-human, non-parenchymal cells such as murine fibroblasts. Use of the culture medium formulation as described herein was found to improve morphology, promote bile canaliculi formation and extend hepatocyte functional lifetime in vitro for over 10 weeks, as compared to only about 3-4 weeks when using a conventional culture medium containing serum from bovine sources. The provided long-term culture model can be used to screen drugs for their efficacious and/or toxic effects over several weeks, improve drug-transporter assays via the larger bile canaliculi network, and to model several chronic liver diseases such as hepatitis, type 2 diabetes, malaria, liver fibrosis, liver cancer, and fatty liver disease

The present disclosure describes the development and uses of the hepatocyte medium described above. The hepatocyte medium comprising human serum contains physiologic levels of insulin and/or glucose thereby designated herein as a “physiologic medium”. The disclosed physiologic medium exhibits numerous benefits relative to traditional medium comprising bovine serum and elevated levels of insulin. The disclosed physiologic medium results in cultures in which hepatocyte morphology is retained at 10 weeks and hepatocyte function (e.g., enzyme activity, urea synthesis, and albumin production) is maintained significantly longer than cultures using bovine serum. Accordingly, the disclosed physiologic medium prolongs the lifetime of hepatocytes in culture relative to medium comprising bovine serum. Further, the disclosed physiologic medium retains hepatocyte polarity and transporters over at least 4 weeks. Importantly, hepatocyte insulin sensitivity is retained in the disclosed physiologically relevant medium. Notably, the hepatocytes cultured in the disclosed physiologic medium retain the ability to correctly identify potential hepatotoxins and non-toxic compounds. While a micropatterned co-culture (MPCC) model was used to demonstrate the effects of the disclosed hepatocyte medium comprising human serum to prolong the hepatocyte lifetime, the medium is broadly applicable to randomly distributed co-cultures of the same two cell types and when stromal cells of other types are used (i.e. endothelia, stellate cells, Kupffer macrophages, other 3T3 clones) in co-culture with hepatocytes. Various aspects of the disclosure are described in more detail below.

Unless otherwise required by context, singular terms as used herein and in the claims shall include pluralities and plural terms shall include the singular. For example, reference to “a cellular island” includes a plurality of such cellular islands and reference to “the cell” includes reference to one or more cells known to those skilled in the art, and so forth.

The use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

Described herein are several definitions. Such definitions are meant to encompass grammatical equivalents.

The term “co-culture” means the growth of more than one distinct cell type in a combined culture. Co-cultures of the present disclosure can include two or more distinct cell types. In some aspects, three or more distinct cell types are included in a co-culture. Co-cultures include, but are not limited to, cultures where two or more cell types are contained in the same container. This includes configurations where one or more of the cell types are contained within a transwell or similar device that is in contact with a container housing one or more cell types.

The term “donor” includes human and other mammalian subjects from which cells such as stem cells, primary endothelial cells, and/or primary hepatocytes may be obtained.

The term “patient” includes human and other mammalian subjects that receive either prophylactic or therapeutic treatment.

The term “population,” when referring to a “cell population,” “population of . . . cells,” and the like, refers to a group of cells of a distinct cell type. The population of cells may contain cells of the same distinct cell type obtained from one or more donors. In other aspects, the population of cells may contain cells of the same distinct type obtained from one or more cell lines.

The term “subject” refers to an animal, including but not limited to a mammal including a human and a non-human primate (for example, a monkey or great ape), a cow, a pig, a cat, a dog, a rat, a mouse, a horse, a goat, a rabbit, a sheep, a hamster, a guinea pig). Preferably, the subject is a human.

### I. Co-Cultures of Human Hepatocytes and at Least One Non-Parenchymal Cell Population

The present disclosure provides a composition comprising a population of human hepatocytes and at least one non-human, non-parenchymal cell population in co-culture in vitro; a culture substrate; and culture medium comprising: human serum, about 0.1 to about 1 nM insulin, and about 1 to about 25 mM glucose. The co-cultures as described herein provide a useful in vitro liver model and thus also provide a unique platform for the development and toxicology screening of therapeutic agents, including high-throughput screening of drug candidates for efficacy and toxicity. The co-cultures described herein include, but are not limited to, random co-cultures and micropatterned co-cultures (“MPCC”). In an aspect, the cell populations are disposed in a micropattern on the culture substrate and the micropattern comprises a predetermined two-dimensional pattern of multiple microdots, the micropattern defined by a microdot diameter and a center-to-center spacing between each of any two neighboring microdots.

The human hepatocytes can be, for example, primary human hepatocytes (“PHHs”), human hepatocytes derived from any human pluripotent stem cells, and an immortalized human hepatocyte cell line. The human hepatocytes may be obtained from a normal human donor or a human donor suffering from a disorder of the liver. Disorders of the liver include, but are not limited to, metabolic disorders such as type 2 diabetes, metabolic syndrome, non-alcoholic fatty liver disease (“NAFLD”), non-alcoholic steatohepatitis (“NASH”), and cardiovascular disease. Disorders of the liver may also include infectious diseases such as hepatitis B, hepatitis C, hepatitis E, dengue fever, and ebola. The present disclosure provides a population of hepatocytes obtained from one or more human donors. A non-limiting, exemplary co-culture is one which includes hepatocytes obtained from one or more human donors suffering from NAFLD or NASH. Another non-limiting, exemplary co-culture is one which includes hepatocytes obtained from one or more normal human donors. Additionally, the human hepatocytes may be derived from any pluripotent stem cells, for example human induced pluripotent stem cells (“iPSC's”), embryonic stem cells (“ESC's”), hepatic resident stem cells (oval cells), and the like. A non-limiting, exemplary co-culture is one which includes hepatocytes derived from human induced pluripotent stem cells such as iCell® Hepatocytes (“iHep” or “iHeps”) available from Cellular Dynamics International of Madison, Wis. The pluripotent stem cell may be from a normal or a diseased donor.

At least one of the non-parenchymal cell populations may be non-human. It is contemplated that in some instances other non-parenchymal cells, both human and non-human, may be included with the disclosed composition. At least one of the non-parenchymal cell populations may comprise stromal cells, such as but not limited to: fibroblasts, fibroblast-derived cells, macrophages, endothelial cells, pericytes, inflammatory cells, cholangiocytes, and other types of stromal cells, and combinations thereof. Fibroblasts may be, for example, mammalian fibroblasts, such as murine embryonic fibroblasts. Non-limiting examples of murine embryonic fibroblasts include 3T3-J2, NIH-3T3, Swiss-3T3, and L1-3T3 murine embryonic fibroblasts. In an aspect, the nonparenchymal cells are 3T3-J2 murine embryonic fibroblasts. A non-limiting, exemplary co-culture is one which includes non-parenchymal cells from normal and diseased donors. Non-parenchymal cells may be obtained from one or more donors suffering from a disorder of the liver. It is contemplated that other non-parenchymal cells, both liver and non-liver, and non-parenchymal cells specifically implicated in a disease can be used to provide an in vitro model for drug testing new drugs to treat the disease. The other non-parenchymal cells, both liver and non-liver, may include non-parenchymal cells that are human or non-human.

The culture substrate may comprise a glass or elastomeric structure with a suitable culture surface, such as a glass, polystyrene, or silicon slide, or polystyrene dish, slide, or microwells. A biopolymer scaffold may optionally be disposed on the culture substrate to further support and promote cell viability. Biopolymers suitable as scaffold material include, but are not limited to, alginate, chitosan, hyaluronate, fibrous proteins, collagen, silk, and elastin. Alternatively, a scaffold may be disposed on the culture substrate that comprises a hydrogel such as collagens, polyacrylamides, polyelectrolyte multilayers, polydimethylsiloxane. In an aspect, a hard substrate may be used.

The co-cultures described herein encompass, but are not limited to, randomly distributed co-cultures of human hepatocytes and non-human, non-parenchymal cells, MPCCs, and hybrids of ECM overlay (“sandwich”) and MPCCs. Co-culturing methods and techniques in general have been described in the literature. In particular, MPCC co-culturing materials, methods and techniques are described detail in Khetani and Bhatia, NATURE BIOTECHNOLOGY, 2008, 26(1): 120-126, the disclosure of which is herein incorporated by reference in its entirety. The co-cultures may be contact models where the human hepatocyte population and at least one non-parenchymal cell population are all contained with the same well. Additionally, small molecules for hepatocyte maturation can also be applied in the co-cultures described herein. Small molecule hepatocyte maturation factors can be any from among those described in the literature and as known to those of skill in the art, including for example any of the small molecules described in Shan et al., NATURE CHEM BIOL. 2013, 9(8): 514-20, the disclosure of which is herein incorporated by reference in its entirety.

Co-cultures of the disclosure may be established as randomly distributed co-cultures of human hepatocytes and non-human, non-parenchymal cells. A co-culture of human hepatocytes and non-human, non-parenchymal cells may be established by seeding both cell populations at the same time. In other aspects, a culture of human hepatocytes may be established first, and then non-human, non-parenchymal cells added.

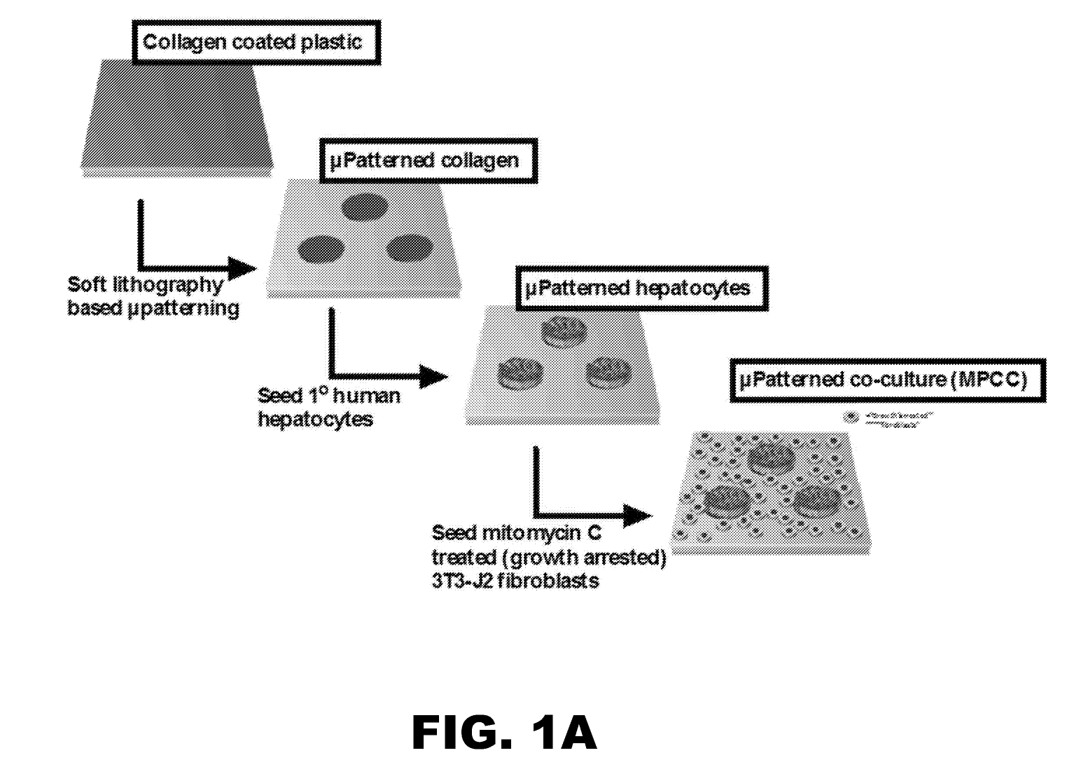

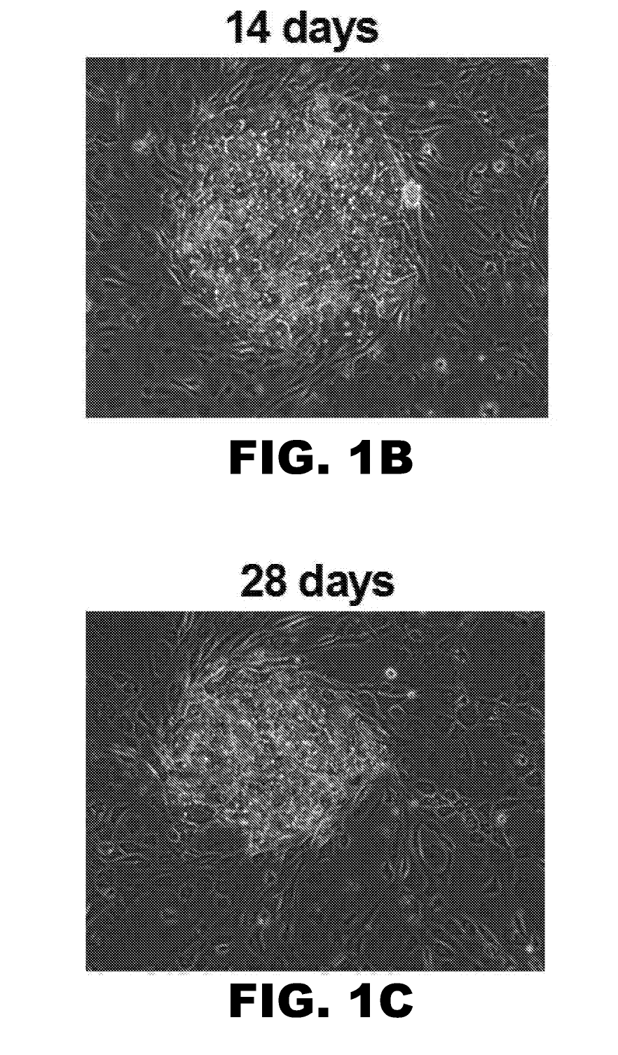

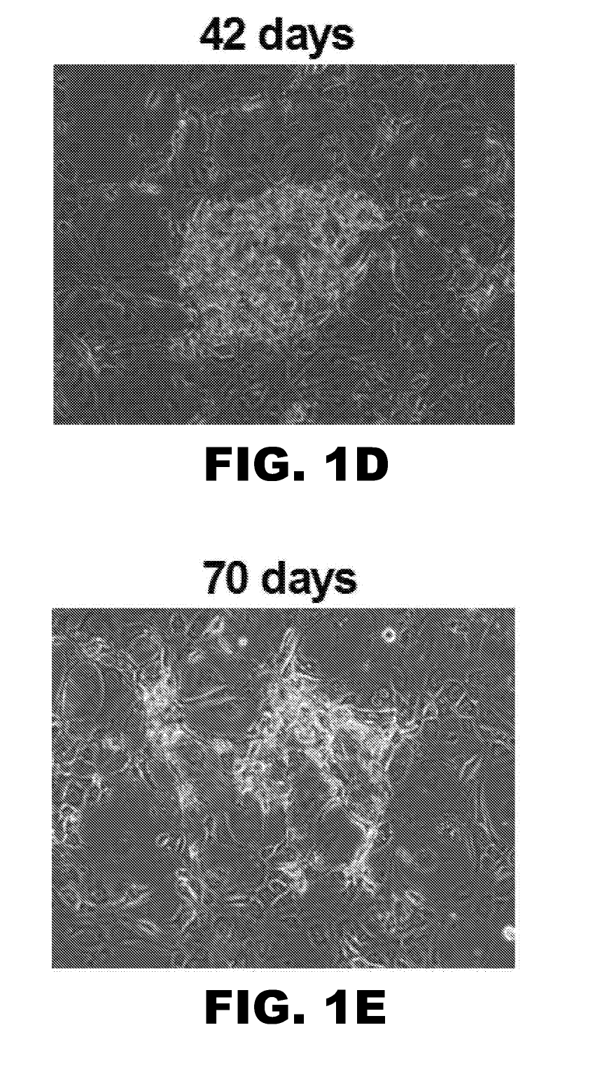

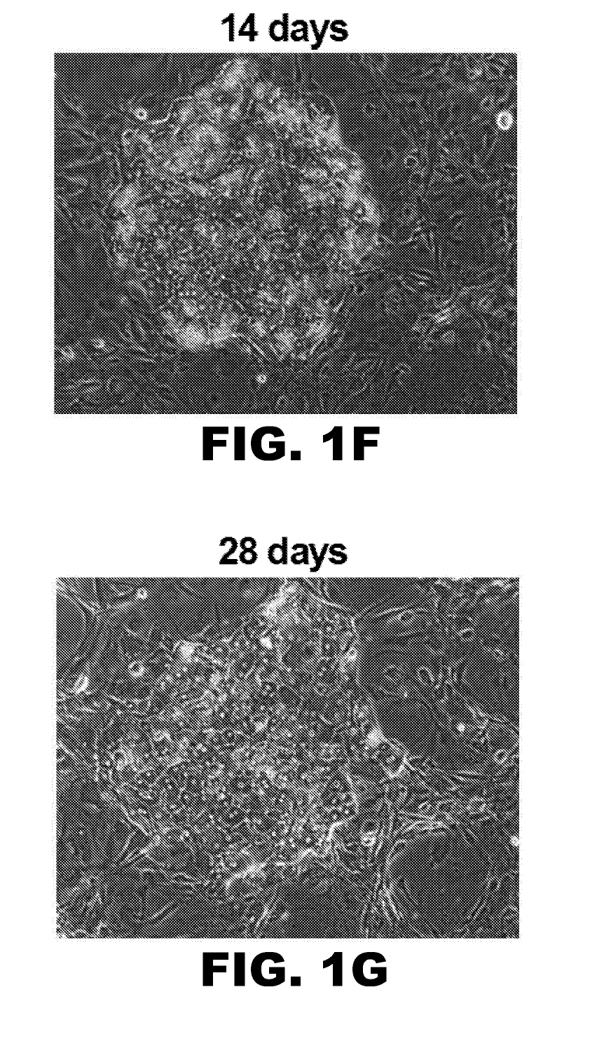

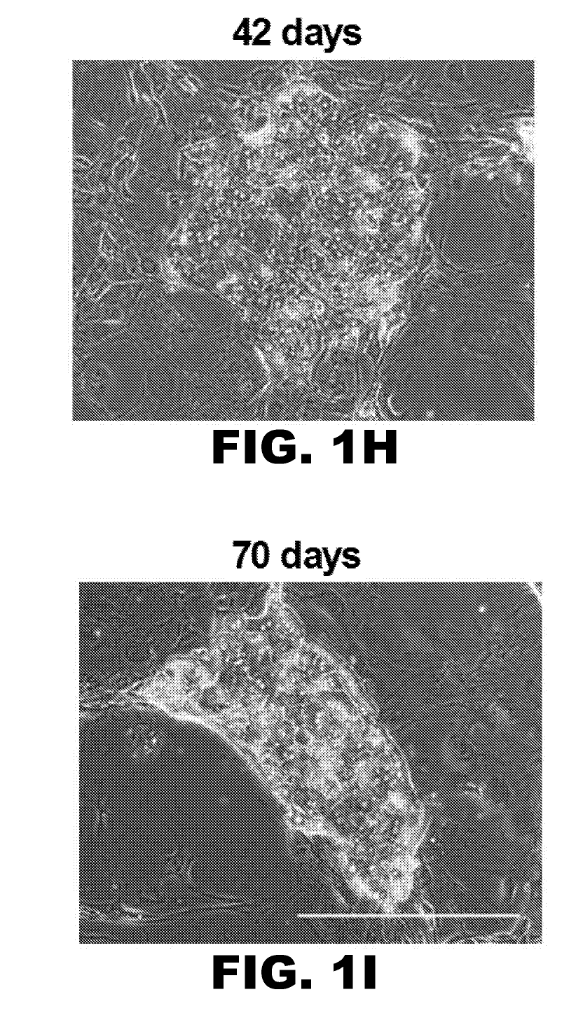

Alternatively, as illustrated in part in FIG. 1A, the co-cultures may be established according to a micropattern established on the culture surface (MPCC). Micropatterning is not required to create co-culture models; however, micropatterning allows for clear demarcation of the hepatocyte islands. The micropattern may comprise for example a predetermined two-dimensional pattern of multiple microdots (“islands”) of the hepatocytes, wherein each microdot has approximately the same microdot diameter and each of any two neighboring microdots shares approximately the same edge-to-edge spacing. While the microdot diameters and microdot spacing may be varied and do vary for cultures with different cell types, it has been found that for hepatocytes, the micropattern may be characterized by microdots each having a diameter of about 500 μm to about 700 μm, and a center-to-center spacing between each microdot of about 700 μm to about 1300 μm, or about 1000 μm to about 1300 μm, including about 1100 μm and about 1200 μm. In some aspects the micropattern may be characterized by microdots each having a diameter of about 500 μm, and an edge-to-edge spacing between each microdot of at least about 700 μm. A micropattern having the foregoing characteristics has been found to result in co-cultures of hepatocytes that remain viable and show evidence of mature phenotype retention for several days and weeks, including up to at least about 8 days, at least about 28 days, and at least 35 days. (U.S. Patent Publication No. 2015/0240203, the disclosure of which is herein incorporated by reference in its entirety.) In another aspect, the micropatterned co-cultures may be established as described in PCT/US2016/039068 and PCT/US2016/045719, both the disclosures of which are herein incorporated by reference in their entirety.

To establish the micropattern, a cell adhesion molecule may be applied to the culture substrate at the microdots, using for example a PDMS stencil. The cell adhesion molecule is any molecule to which the hepatocytes selectively adhere relative to inter-microdot space, such as collagen, fibronectin, vitronectin, laminin, extracellular matrix proteins, Arg-Gly-Asp (RGD) peptide, Tyr-Ile-Gly-Ser-Arg (YIGSR) peptide, glycosaminoglycans, hyaluronic acid, integrins, ICAMs, selectins, cadherins, cell surface protein-specific antibodies, any combination thereof, and any composition composed substantially of purified extracellular matrix protein, or mixtures of extracellular matrix proteins. Suitable extracellular matrix can be provided for example by ECM derived directly from mammalian liver, such as porcine or human liver. In one micropatterned hepatocyte co-culture, the cell adhesion molecule is, for example, any of the many extracellular matrix protein products available from a variety of commercial suppliers. Some non-limiting examples of extracellular matrix protein products available from commercial suppliers include rat tail collagen 1, matrigel, human collagens 1, 3 or 4, fibronectin, laminin and decorin, available from suppliers such as Corning Life Sciences, R& D Systems, Thermo-Fisher, and VWR. In another micropatterned hepatocyte co-culture, the cell adhesion molecule is, for example, a commercially available collagen, such as rat tail collagen.

In both the randomly distributed co-cultures and MPCCs, the hepatocytes are seeded onto the culture substrate and allowed to attach. In general, the attachment of hepatocytes to substrate occurs in the absence of serum (i.e. culture medium not supplemented with serum). The hepatocytes may be allowed to attach for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In an aspect, the hepatocytes are allowed to attach for about 3 hours to about 24 hours. In another aspect, the hepatocytes are allowed to attach for about 4 hours to about 5 hours. In another aspect, the hepatocytes are allowed to attach for about 16 hours to about 18 hours. Following attachment, the hepatocytes are incubated in medium comprising fetal bovine serum to allow the hepatocytes to spread. The hepatocytes may be incubated in medium comprising fetal bovine serum for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In an aspect, the hepatocytes are incubated in the medium comprising fetal bovine serum for about 16 to about 24 hours.

Following seeding of the hepatocytes onto the culture substrate, the non-human, non-parenchymal cell population may be seeded onto the culture substrate. Prior to seeding of the non-parenchymal cell population, the non-parenchymal cell population may be expanded in the presence of medium comprising bovine serum. Upon seeding of the non-parenchymal cell population onto the culture substrate comprising hepatocytes, the hepatocytes and non-parenchymal cell population may be incubated in the presence of medium comprising bovine serum and/or fetal bovine serum until the non-parenchymal cells are confluent. In an aspect, the bovine serum is calf (12-18 month) serum. For example, the hepatocytes and non-parenchymal cell population may be incubated in the presence of medium comprising bovine serum for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 26 hours, about 28 hours, about 30 hours, about 32 hours, about 34 hours, about 36 hours, about 42 hours, about 48 hours, about 54 hours, about 60 hours, about 66 hours, about 72 hours, about 78 hours, or about 84 hours. In an aspect, the hepatocytes and non-parenchymal cell population may be incubated in the presence of medium comprising bovine serum for about 24 hours to about 72 hours.

Following the establishment of confluence of the hepatocytes and non-human, non-parenchymal cell population, the hepatocytes and non-human, non-parenchymal cell population are cultured in the presence of medium comprising human serum. In some aspects the medium may further comprise insulin and/or glucose. In an aspect, the hepatocytes and non-human, non-parenchymal cell population are cultured in the presence of physiologic medium. As used herein, “physiologic medium” contains human serum and physiologic levels of insulin and/or glucose. In certain aspects, the non-parenchymal cells are murine fibroblasts. Prior to the experiments disclosed herein, it was unknown that murine fibroblasts would survive and grow in human serum. In an aspect, the hepatocytes and non-parenchymal cell population are cultured in the presence of medium comprising human serum, about 0.1 nM to about 1 nM insulin, and about 1 mM to about 25 mM glucose. The base medium can be Dulbecco's modified Eagle's medium (DMEM) or William's E base. The human serum may be obtained from a normal human donor or a human donor suffering from a disorder of the liver. The human serum can be obtained from one or more human donors. The culture medium comprises from about 1% vol/vol to about 100% vol/vol human serum, from about 1% vol/vol to about 90% vol/vol human serum, from about 1% vol/vol to about 80% vol/vol human serum, from about 1% vol/vol to about 70% vol/vol human serum, from about 1% vol/vol to about 60% vol/vol human serum, from about 1% vol/vol to about 50% vol/vol human serum, from about 1% vol/vol to about 40% vol/vol human serum, from about 1% vol/vol to about 30% vol/vol human serum, from about 1% vol/vol to about 20% vol/vol human serum, from about 1% vol/vol to about 15% vol/vol human serum, from about 1% vol/vol to about 10% vol/vol human serum, from about 5% vol/vol to about 20% vol/vol human serum, from about 5% vol/vol to about 15% vol/vol human serum, or from about 5% vol/vol to about 10% vol/vol human serum. The culture medium can comprise about 5% vol/vol, about 6% vol/vol, about 7% vol/vol, about 8% vol/vol, about 9% vol/vol, or about 10% vol/vol human serum. Specifically, the culture medium comprises about 10% vol/vol human serum. Further, the culture medium comprises physiologic levels of insulin. In an aspect, the culture medium comprises from about 0.1 nM to about 1 nM of insulin. In another aspect, the culture medium comprises from about 0.1 nM to about 100 nM of insulin, from about 0.1 nM to about 10 nM of insulin, from about 0.1 nM to about 1 nM of insulin, from about 0.5 nM to about 100 nM of insulin, from about 0.5 nM to about 10 nM of insulin, or from about 0.5 nM to about 1 nM of insulin. The culture medium can comprise about 0.1 nM, about 0.5 nM, about 1 nM, or about 10 nM insulin. Specifically, the culture medium comprises about 0.5 nM insulin. Still further, the culture medium comprises physiologic levels of glucose. Accordingly, the culture medium comprises from about 1 mM to about 25 mM of glucose, from about 1 mM to about 20 mM of glucose, from about 1 mM to about 15 mM of glucose, from about 1 mM to about 10 mM of glucose, from about 1 mM to about 5 mM of glucose, from about 5 mM to about 25 mM of glucose, from about 5 mM to about 20 mM of glucose, from about 5 mM to about 15 mM of glucose, or from about 5 mM to about 10 mM of glucose. The culture medium can comprise about 1 mM, about 5 mM, about 10 mM, about 15 mM, about 20 mM, or about 25 mM glucose. Specifically, the culture medium comprises about 5 mM glucose.

The culture medium may be supplemented with various other components known to facilitate growth of cells, specifically hepatocytes. In an aspect, the culture medium further comprises L-glutamine, transferrin, selenium, dexamethasone, glucagon, and/or a buffer such as HEPES. The culture medium may comprise about 1 mM to about 20 mM L-glutamine, or about 1 mM to about 10 mM L-glutamine, or about 1 mM to about 5 mM L-glutamine, or about 1 mM to about 4 mM L-glutamine, or about 4 mM L-glutamine. Further, the culture medium may comprise about 1 μg/mL to about 10 μg/mL transferrin, or about 3 μg/mL to about 8 μg/mL transferrin, or about 5 μg/mL to about 7 μg/mL transferrin, or about 6 μg/mL transferrin. Additionally, the culture medium may comprise about 1 ng/mL to about 10 ng/mL selenium, or about 3 ng/mL to about 8 ng/mL selenium, or about 5 ng/mL to about 7 ng/mL selenium, or about 6 ng/mL selenium. Still further, the culture medium may comprise about 1 nM to about 1000 nM dexamethasone, or about 10 nM to about 1000 nM dexamethasone, or about 100 nM to about 1000 nM dexamethasone, or about 250 nM to about 750 nM dexamethasone, or about 500 nM dexamethasone. In addition, the culture medium may comprise about 1 nM to about 10 nM glucagon, or about 1 nM to about 5 nM glucagon, or about 2 nM glucagon. The culture medium may also comprise about 1 mM to about 50 mM of a buffer such as HEPES, or about 5 mM to about 25 mM of a buffer such as HEPES, or about 10 mM to about 20 mM of a buffer such as HEPES, or about 15 mM of a buffer such as HEPES.

In an aspect, the culture medium comprises Dulbecco's modified Eagle's medium (DMEM) or William's E base, from about 1% to about 20% vol/vol human serum, from about 0.1 nM to about 10 nM of insulin, from about 1 mM to about 25 mM of glucose, and L-glutamine, transferrin, selenium, dexamethasone, glucagon, and a buffer such as HEPES. In another aspect, the culture medium comprises Dulbecco's modified Eagle's medium (DMEM) or William's E base, from about 1% to about 20% vol/vol human serum, from about 0.1 nM to about 10 nM of insulin, from about 1 mM to about 25 mM of glucose, about 1 mM to about 20 mM L-glutamine, about 1 μg/mL to about 10 μg/mL transferrin, about 1 ng/mL to about 10 ng/mL selenium, about 1 nM to about 1000 nM dexamethasone, about 1 nM to about 10 nM glucagon, and about 1 mM to about 50 mM of a buffer such as HEPES. In still another aspect, the culture medium comprises Dulbecco's modified Eagle's medium (DMEM) or William's E base, from about 5% to about 10% vol/vol human serum, from about 0.5 nM to about 1 nM of insulin, from about 5 mM to about 10 mM of glucose, about 1 mM to about 4 mM L-glutamine, about 5 μg/mL to about 7 μg/mL transferrin, about 5 ng/mL to about 7 ng/mL selenium, or about 250 nM to about 750 nM dexamethasone, about 1 nM to about 5 nM glucagon, and about 10 mM to about 20 mM of a buffer such as HEPES. In still yet another aspect, the culture medium comprises Dulbecco's modified Eagle's medium (DMEM) or William's E base, about 10% vol/vol human serum, about 0.5 nM insulin, about 5 mM glucose, about 4 mM L-glutamine, about 6 μg/mL transferrin, about 6 ng/mL selenium, about 500 nM dexamethasone, about 2 nM glucagon, and about 15 mM of a buffer such as HEPES.

The co-cultures described herein may be maintained in vitro in the presence of culture medium comprising human serum, insulin and glucose for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 30 hours, at least 36 hours, at least 42 hours, at least 48 hours, at least 54 hours, or at least 60 hours. In another aspect, the co-cultures described herein may be maintained in vitro in the presence of culture medium comprising human serum, insulin and glucose for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days. In still another aspect, the co-cultures described herein may be maintained in vitro in the presence of culture medium comprising human serum, insulin and glucose for at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, or more than 31 days. In still yet another aspect, the co-cultures described herein may be maintained in vitro in the presence of culture medium comprising human serum, insulin and glucose for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. Specifically, the co-cultures described herein may be maintained in vitro in the presence of culture medium comprising human serum, insulin and glucose for 4 weeks, 5 weeks, 6 weeks, 7 weeks, 8 weeks, 9 weeks, or 10 weeks. Notably, the co-cultures described herein may be maintained in vitro in the presence of culture medium comprising human serum, insulin and glucose for 6 weeks, 7 weeks, 8 weeks, 9 weeks, or 10 weeks.

The co-culture maintained in the presence of culture medium comprising human serum, insulin and glucose may be used for drug discovery and drug screening as described in greater detail below. When contacting the co-culture with a test compound, the culture medium comprising human serum, insulin and glucose may be removed and a medium comprising a test compound with no human serum, various amounts of human serum or 100% human serum may be added. The medium may comprise test compound with about 1% to about 100% vol/vol human serum. For example, the medium may comprise test compound with about 1% to about 10% vol/vol, about 1% to about 20% vol/vol, about 1% to about 40% vol/vol, about 1% to about 60% vol/vol, about 1% to about 80% vol/vol, about 20% to about 40% vol/vol, about 20% to about 60% vol/vol, about 20% to about 80% vol/vol, about 20% to about 100% vol/vol, about 40% to about 60% vol/vol, about 40% to about 80% vol/vol, about 40% to about 100% vol/vol, about 60% to about 80% vol/vol, about 60% to about 100% vol/vol, or about 80% to about 100% vol/vol human serum. If human serum is present, binding of test compound to proteins found in human serum must be accounted for. Methods of doing so are known to those of skill in the art.

The present disclosure also provides a kit for determining the effect of a test agent on hepatocytes. A kit may comprise for example: a population of human hepatocytes and at least one non-human, non-parenchymal cell population for preparing one or more MPCC's as disclosed herein. In one aspect, the hepatocytes may be obtained from one or more human donors suffering a disorder of the liver. The kit may further comprise a culture medium as described herein, and/or additional materials or reagents for testing various biological activities of the cells in culture. For example, the kit may contain separately packaged amounts of a glucose-free medium, human serum, glucose, insulin, L-glutamine, transferrin, selenium, glucagon, dexamethasone, HEPES, a stain or dye such as but not limited to a fluorimetric dye, a lipid dye such as Nile red, and/or a cellular stain for glycogen such as PAS stain. The kit may further comprise one or more culture substrates such as a glass, silicon, or polystyrene slide or culture well, and an amount of a cell adhesion molecule. The cell adhesion molecule may be disposed according to a micropattern on the culture substrate as described herein above. Alternatively, the kit may provide an amount of the cell adhesion molecule and a PDMS stencil which can be used together to establish a micropattern as described herein on the culture substrate.

The kit may further comprise a reporter molecule or label capable of generating a signal indicative of a level of a cellular activity of interest in the hepatocytes, such as but not limited a vital dye, a lipid dye, a colorimetric agent, or a bioluminescent marker. The kit may include a detectable label such as a fluorophore, a radioactive moiety, an enzyme, a chromophore, a chemiluminescent label, or the like, and/or reagents for carrying out detectable labeling. The labels and/or reporter molecules, any calibrators and/or controls can be provided in separate containers or pre-dispensed into an appropriate assay format, for example, into microtiter plates.

It is contemplated for example that one or more of the presently disclosed co-cultures can be provided in the form of a kit with one or more containers such as vials or bottles, with each container containing a separate population of cells and/or reagents and washing reagents employed in an assay. The kit can comprise at least one container for conducting the assay, and/or a buffer, such as an assay buffer or a wash buffer, either one of which can be provided as a concentrated solution, a substrate solution for the detectable or a stop solution. The kit may comprise all components, e.g., reagents, standards, buffers, diluents, etc., which are necessary to perform the assay. The kit may contain instructions for determining the presence or amount of any metabolite, biomarker, label, or reporter of interest in the co-culture, in paper form or computer-readable form, such as a disk, CD, DVD, or the like, and/or may be made available online.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, enzyme substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. Where appropriate, the kit optionally also can contain reaction vessels, mixing vessels, and other components that facilitate the preparation of reagents or the test sample. The kit can also include one or more instruments for assisting with obtaining a test sample, such as a syringe, pipette, forceps, measured spoon, or the like.

### II. Uses

The hepatocyte co-cultures as described herein may be used in various methods, such as but not limited to drug discovery and drug screening. Such co-culture systems can be used to develop and screen candidate therapeutic agents for treating any hepatic disease or disorder, or for screening the toxicity of candidate therapeutic agents for treating any other disease or disorder. In an aspect, the hepatic disease or disorder is metabolic disorder such as type 2 diabetes, metabolic syndrome, non-alcoholic fatty liver disease (“NAFLD”), non-alcoholic steatohepatitis (“NASH”), and cardiovascular disease or an infectious disease such as hepatitis B, hepatitis C, hepatitis E, dengue fever, and ebola. For example, co-cultures as described herein provide for a model of in vitro prediction of drug induced liver toxicity, and establish a new direction in in vitro models of the liver. Notably, the physiologic hepatocyte medium provided herein facilitates the use of in vitro cultures for at least 4 weeks, at least 5 weeks, at least 6 weeks, at least 8 weeks, or at least 10 weeks. The cultured hepatocytes have a significantly prolonged lifetime and maintain function without losing their utility for drug screening and glucose metabolism studies.

A candidate therapeutic agent (also referred to as a “drug candidate” or “test compound”) may be a small molecule, a peptide, a polypeptide, an oligonucleotide, a polynucleotide, or an antibody.

Any of the methods may involve determining a baseline or control value, for example, of any indicator of liver function such as gluconeogenesis, glycolysis, glycogen storage, enzyme activity, albumin secretion, urea production, gene expression, inducible liver enzyme activity and the like, in the hepatocytes in co-culture before administering a dosage of a candidate therapeutic agent or other test agent, and comparing this with a value or level after the exposure and noting any significant change (i.e., greater than the typical margin of experimental error in repeat measurements of the same sample, expressed as one standard deviation from the mean of such measurements) over the control. In a non-limiting example, at least one indicator of hepatic function can be a measure of albumin production, urea production, ATP production, glutathione production, enzyme activity, lipid accumulation, liver gene expression, liver protein expression, or inducible liver enzyme activity in the hepatocytes.

### (a) Screening Assays

The present disclosure provides an in vitro model of diseases or disorders of the liver which can be utilized in various methods for identifying and screening of potential therapeutic agents, and for drug development. Disorders of the liver include, but are not limited to, metabolic disorders such as type 2 diabetes, metabolic syndrome, non-alcoholic fatty liver disease (“NAFLD”), and non-alcoholic steatohepatitis (“NASH”). In an aspect, the hepatic disease or disorder is an infectious disease, such as hepatitis B, hepatitis C, hepatitis E, dengue fever, and ebola.

For example, the compositions of the present disclosure may be used in vitro to screen a wide variety of compounds, such as small molecules, antibodies, peptides, polypeptides, nucleic acid-based agents and the like, to identify therapeutic agents having a therapeutic effect on liver function in any disease or disorder of the liver, and/or to assess the toxicity of any such therapeutic agent before clinical implementation. For example, following contact of a co-culture with a candidate therapeutic agent, various cellular functions in the hepatocytes and/or human endothelial cells may be assessed by examining gene expression, albumin production, urea production, cytochrome P450 (CYP) metabolic activity or any inducible liver enzyme activity, organelle stress, cell surface markers, secreted factors, uptake and secretion of liver-specific products, and response to hepatotoxins, by detecting and/or measuring level of a protein, metabolite, reporter molecule, label, or gene expression level such as through gene fluorescence in the cell or in the culture media. In non-limiting example, at least one indicator of hepatic function can be, for example, albumin production, urea production, ATP production, glutathione production, enzyme activity, lipid accumulation, liver gene expression, or liver protein expression in the hepatocytes. Additionally, following contact of a co-culture with a candidate therapeutic agent, the co-cultures may be used to determine the hepatic clearance of a potential therapeutic agent. Accordingly, the co-cultures may be used to quantitatively determine hepatic clearance rates of drugs. See for example, Lin et al., Drug Metab Dispos 2016; 44(1): 127-36, the disclosure of which is hereby incorporated by reference in its entirety. Further, following contact of a co-culture with a candidate therapeutic agent, the co-cultures may be used to determine drug metabolites. Accordingly, using the co-cultures disclosed herein, metabolite profile data may be obtained for a potential therapeutic agent. See for example, Wang et al., Drug Metab Dispos 2010; 38(10): 1900-5, the disclosure of which is hereby incorporated by reference in its entirety.

Gluconeogenesis and other liver functions such as albumin secretion, urea production, and glycolysis and glycogen storage may be monitored in the presence and absence of one or more stimuli, test agent, or candidate therapeutic agent. For example, hepatocytes in co-culture as described herein may be tested for any one or more of albumin secretion, urea production, ATP production, lipid accumulation, induction of inducible liver (e.g., CYP) enzyme levels, gluconeogenesis, glycolysis and glycogen storage in the presence and absence of varying levels of candidate therapeutic agents. In any method involving measurement of one or more inducible liver enzymes, such enzymes include, in non-limiting example, CYP enzymes such as CYP2C9 (luciferin-H), CYP3A4 (luciferin-IPA), CYP1A1, CYP1A2, CYP2B6, CYP2A6, and CYP2D6 (luciferin ME-EGE), all CYP450 enzymes such as CYP2C8, CYP2C19, CYP2E1, and phase II enzymes such as UGTs, SULTs and NATs, and any combination thereof.

Levels of biomarkers such as for example specific metabolites may also be used in screening assays for agents. This may also be accomplished using standard cytological and/or histological techniques including the use of immunocytochemical techniques employing stains that recognize specific cellular components such as lipids, or antibodies that specifically bind to biomarkers with antigenic activity. For example, co-cultures of the present disclosure may be exposed to a test agent or candidate therapeutic agents. After incubation, the co-cultures may be examined for change in biomarker production as an indication of the efficacy of the test substance. Varying concentrations of a candidate therapeutic agent may be tested as known in the art to derive a dose-response curve.

### (b) Target Validation

The compositions of the disclosure can be used in drug development for specific target identification and target validation. The disclosed co-cultures are useful for identifying targets and predicting the role of one or more biomolecules in liver function in a disease or disorder of the liver. A “disease or disorder of the liver” is any medical condition having a negative effect on any liver function. Non-limiting examples of liver diseases and disorders include cirrhosis, diabetes, fibrosis, any chronic hepatitis (including but not limited to A, B, C, D, and E), non-alcoholic fatty liver disease (“NAFLD”), alcoholic fatty liver, tumors of the liver such as hepatic carcinoma, and genetic disorders such as alpha-1-anti-trypsin deficiency.

For example, the cultures and systems may be used to identify proteins playing a potential role in fibrosis of the liver, or those playing a potential role in diabetic processes or diabetic liver pathways. Specifically, given that the disclosed physiologic hepatocyte medium results in decreased insulin resistance of hepatocytes in culture, various factors that are implicated in insulin resistance development may be supplemented in the medium to identify the mechanisms behind disease progression. Identified proteins may be modulated (e.g., up-regulated or down-regulated) in the co-cultures described herein, and processes and pathways related to diabetes may be assayed following modulation.

The co-cultures and systems are also useful for validating the predicted role of one or more biomolecules in liver function in a disease or disorder of the liver. For example, proteins identified in preliminary studies (e.g., studies of primary hepatocytes in conventional culture systems or cryogenically preserved hepatocytes, studies in other liver models, differential expression studies, etc.) as playing a potential role in disease processes or disease pathways can be tested in a composition as described herein to confirm the potential role. Proteins identified from preliminary studies, for example proteins suspected to play a role in diseased or disordered liver function, may be modulated (e.g., up-regulated or down-regulated) in the co-cultures described herein, and processes and pathways related to the disease or disorder may be assayed following modulation. For example, candidate proteins can be “knocked out/down” using gene knockout or suppression techniques, for example, using various genomic editing techniques, or the introduction of RNA interference (RNAi) agents. Inhibition of liver pathways may be tested following down-regulation and candidate proteins thought to be important in disease or disordered liver function may be thus validated.

Any method using the co-cultures as disclosed herein may comprise initially preparing or otherwise obtaining a co-culture of hepatocytes and non-human, non-parenchymal cells as described herein. In one aspect, a stable, growing co-culture is established as described herein above. In one aspect, the present disclosure provides a method of determining the efficacy of a candidate therapeutic agent for treating a disease or disorder of the liver. The candidate therapeutic agent may be a small molecule, a peptide, a polypeptide, an oligonucleotide, a polynucleotide, or an antibody.

The co-culture is exposed to varying concentrations of the candidate therapeutic agent. The amount of the candidate therapeutic agent may be, according to knowledge available to those of skill in the art, an amount representing a proposed dose or range of proposed doses in a clinical population. The time over which the hepatocytes in the co-culture are exposed to the candidate therapeutic agent may be, according to knowledge available to those of skill in the art, a period of days, weeks, or months representing time course of exposure in a clinical population. After incubation with the agent, the culture is examined to determine impact of the agent if any on one or more target biomolecules or pathways identified as potentially involved in liver function in a disease or disorder of the liver, as described above. Once a testing range is established, varying concentrations of the agent can be tested to determine therapeutically effective amount of the test compound.

As noted above, the human hepatocytes can be obtained or derived from human donors. In further aspects, human hepatocytes can be derived from stem cells obtained from one or more human donors. In another aspect, the human hepatocytes can be obtained from one or more human donors suffering from a disease or disorder of the liver. Alternatively, the human hepatocytes can be derived from stem cells obtained from one or more human donors suffering from a disease or disorder of the liver.

By way of example, human hepatocytes can be obtained from one or more human donors suffering from a metabolic disorder of the liver. The methods therefore encompass, for example, a method for testing a candidate therapeutic agent for treating a disorder of the liver, including maintaining a co-culture as described herein for a time and under conditions sufficient to allow glucose production by the hepatocytes; and determining a level of glucose production by the hepatocytes, wherein the level of glucose production relative to the level of glucose production in a population of control hepatocytes is indicative of the efficacy of the test compound as an therapeutic agent for treating the disorder of the liver. The method may further comprise, prior to determining the level of glucose production by the hepatocytes: depleting the co-culture of glycogen in glucose-free medium for a period of at least about 12 hours; contacting the co-culture with at least one substrate of a gluconeogenesis enzyme; and maintaining the co-culture for a period of at least about 12 hours under conditions sufficient for glucose production in the hepatocytes to occur. The co-culture may be maintained for a period of at least about 24 hours, or at least 48 hours under conditions sufficient for glucose production in the hepatocytes to occur. The at least one substrate of a gluconeogenesis enzyme may be for example lactate or pyruvate.

It should be understood that the present disclosure encompasses methods of identifying any test agent useful for modulating a biological activity of interest in a hepatocyte, in which a co-culture as disclosed herein is contacted with the test agent; the co-culture is maintained for a time and under conditions sufficient for the hepatocytes to generate a signal indicative of the biological activity; and a signal generated by the hepatocytes in the presence of the test agent is detected, wherein the signal relative to a signal generated in a control hepatocyte in a control co-culture is indicative of an effect on the biological activity of interest in the hepatocytes. The signal indicative of the biological activity of interest may be for example a protein expression level or a protein secretion level. The biological activity of interest may be glucose metabolism. The biological activity of interest may be albumin secretion or urea synthesis.

### (c) Toxicity Studies

In addition to the above-described uses of the cultures and/or systems of the disclosure in screening for therapeutic agents for treating a disease or disorder of the liver, the co-cultures may also be used in toxicology studies to determine the hepatotoxicity of an agent identified as a potential therapeutic agent. Toxicology studies may be performed on co-cultures featuring hepatocytes from human donors suffering from a disease or disorder of the liver, as described herein, which may be contrasted with comparable studies in cells from a different source. The co-cultures described herein may be used in vitro to test a variety of potential therapeutic compounds for hepatotoxicity. Any of the screening methods described herein above may further comprise determining the toxicity of the agent by measuring in the hepatocytes at least one cell signal indicative of cell toxicity. Still further, the co-cultures may be used to determine the hepatic clearance of a potential therapeutic agent. Accordingly, the co-cultures may be used to quantitatively determine hepatic clearance rates of drugs. Additionally, the co-cultures may be used to determine drug metabolites. Accordingly, using the co-cultures disclosed herein, metabolite profile data may be obtained for a potential therapeutic agent.

Toxicity results may be assessed for example by observation of any of the following: a change in albumin and/or urea production, induction of any inducible liver enzyme such as cytochrome P450 (CYP) enzymes, cellular necrosis, loss of membrane integrity, cell lysis, decrease in cell viability, apoptosis, mitochondrial membrane potential, mitochondrial DNA, ER stress, organelle stress, change in endothelial cell surface markers, secretion of cell factors, and steatosis, using any one or more of vital staining techniques, ELISA assays, RT-qPCR, immunohistochemistry, imaging, electron microscopy, and the like or by analyzing the cellular content of the culture, e.g., by total cell counts, and differential cell count, by metabolic markers such as MTT and XTT, or by hepatocyte imaging technology (HIAT).

For example, co-cultures as described herein are exposed to varying concentrations of a candidate therapeutic agent. The amount of the candidate therapeutic agent may be, according to knowledge available to those of skill in the art, an amount representing a proposed dose or range of proposed doses in a clinical population. The time over which the hepatocytes are exposed to the candidate therapeutic agent may be, according to knowledge available to those of skill in the art, a period of days, weeks, or months representing time course of exposure in a clinical population. After incubation with the agent, the culture is examined to determine the highest tolerated dose, i.e., the concentration of the agent at which the earliest morphological and/or functional abnormalities appear or are detected. Cytotoxicity testing may also be performed using a variety of supravital dyes to assess cell viability in the culture system, using techniques known to those skilled in the art. Once a testing range is established, varying concentrations of the agent can be examined for hepatotoxic effect.

The present disclosure thus provides a method for determining the cellular toxicity of a candidate therapeutic agent or test compound, the method comprising contacting a co-culture as described herein with the test compound; maintaining the co-culture for a time and under conditions sufficient to allow an effect of the test compound on the hepatocytes; and taking a test measurement and/or otherwise obtaining test data indicative of a negative impact of the test compound on hepatocytes, which is indicative of toxicity of the test compound. The test measurement can be any measurement which provides an indicator of hepatic cell function. In a non-limiting example, at least one indicator of hepatic function can be a measure of albumin production, urea production, ATP production, glutathione production, enzyme activity, lipid accumulation, liver gene expression, liver protein expression, or inducible liver enzyme activity in the hepatocytes. For example, the test measurement can be a measurement of at least one or any combination of albumin, urea, enzyme activity, lipid accumulation, ATP production, and gene expression. The test measurement can be a measurement of at least one inducible liver (e.g., CYP) enzyme level. Test data may include applying hepatocyte imaging technology (HIAT) to the hepatocytes and/or human endothelial cells to obtain a test image. Test data may include using other imaging technology on the co-cultures to obtain a test image. The test measurement and/or test image is compared to a control measurement or control image from the hepatocytes before contact with the test compound, and a difference between the test measurement and control measurement, or between test image and control image is indicative of toxicity of the test compound. For example, a relative decrease in albumin and/or urea production in test measurements as compared to control, following exposure of the co-culture to the test compound is indicative of hepatotoxicity.

The present disclosure also provides a method of determining the toxicity arising from a drug interaction. For example, the potential toxicity of an interaction between a first test compound and a second test compound can be examined by contacting a co-culture as described herein with the first and second test compounds; maintaining the co-culture for a time and under conditions sufficient to allow an effect of an interaction between the first and second test compounds on the hepatocytes; and taking a test measurement and/or otherwise obtaining test data as described above, which is indicative of toxicity of the interaction of the first and second test compounds.

The present disclosure also provides a method of determining whether a test compound alleviates hepatic dysfunctions caused by hepatocytes. For example, the effects of a test compound can be examined by contacting a co-culture as described herein with the test compound; maintaining the co-culture for a time and under conditions sufficient to allow an effect of the test compound on the hepatocytes; and taking a test measurement and/or otherwise obtaining test data as described above, which is indicative of effect of test compounds on hepatic function. In some aspects more than one test compound can be examined at one time. For example, two, three, four, five, six, 7, 8, 9, or 10 test compounds can be examined.

Additionally, the present disclosure thus also provides a method for determining the effects of chronically elevated or reduced levels of glucose, fructose and/or fatty acids on the liver and liver function. The method comprises for example contacting a co-culture as described herein with a predetermined amount of one or more metabolites such as glucose, fructose, and or fatty acids, wherein the hepatocytes are obtained from one or more human donors suffering from a disorder of the liver; maintaining the co-culture for a time and under conditions sufficient for the hepatocytes to generate a signal indicative of modified cellular function induced by the predetermined amount of one or more metabolites; and detecting the signal generated by hepatocytes in the presence of the one or more metabolites, wherein the signal relative to a signal generated in a control cell subject to the same conditions is indicative of an effect of the amount of the one or more metabolites on the hepatocytes. The signal indicative of an effect on cell function may be a change in transcription, translation or secretion of a protein, cellular necrosis, loss of membrane integrity, cell lysis, decrease in cell viability, apoptosis, mitochondrial membrane potential, mitochondrial DNA, ER stress, and steatosis. The predetermined amount may be an amount which is elevated or reduced relative to a control amount which is representative of an amount of each metabolite which is considered within the range of normal in vivo values for the metabolite. The time over which the hepatocytes are exposed to the elevated or reduced level(s) of metabolite(s) may be, according to knowledge available to those of skill in the art, a period of days, weeks or months representing chronic elevation or reduction of the metabolite in a clinical population.

It should be understood that many other signals of toxicity from the hepatocytes can be detected and/or measured and compared to controls to identify and/or quantify toxicity induced by a candidate therapeutic agent, wherein the signal relative to a signal generated in a control co-culture is indicative of a toxic effect of the candidate agent on the hepatocytes. Such signals include, in non-limiting example, cellular necrosis, loss of membrane integrity, cell lysis, decrease in cell viability, apoptosis, mitochondrial membrane potential, mitochondrial DNA, ER stress, and steatosis, any one of which can be readily measured using techniques and materials known in the art.

### (d) Personalized Medicine

It has been found that certain correlations can exist between an individual subject's particular genotype with respect to specific molecular markers, and drug treatment efficacy. Any of the co-cultures and methods described herein can also be used to develop personalized medicine, to determine whether any such correlation exists between a particular genotype and selected drug treatment for a disease or disorder of the liver. For example, co-cultures can be prepared using human hepatocytes derived from pluripotent stem cells obtained from a variety of donors of different genotypes, and any therapeutic candidate can be tested for efficacy against each genotype to determine whether any one or subset of the tested genotypes fares better or worse with a given therapeutic candidate. Any therapeutic candidate can be tested for effect on any inducible liver enzymes, and/or for a negative interaction with a second therapeutic candidate. Such information considered together with the genotype of an individual patient, can be used by a health care provider to determine a treatment option with the highest likelihood of efficacy for the individual subject, and/or to determine a risk of a negative side effect in the individual subject from a therapeutic candidate.

### (e) Regenerative Medicine

The present disclosure also provides methods of engineering liver tissue for regenerative medicine. The liver tissue prepared via the methods disclosed herein may be used to replace failing livers. The use of the physiologic hepatocyte medium comprising human serum greatly enhances the stabilization of liver cells prior to implantation. Further, the medium prevents the liver cells from developing disease phenotypes prior to implantation.

It will be readily apparent to those skilled in the art that other suitable modifications and adaptations of the methods of the disclosure described herein are obvious and may be made using suitable equivalents without departing from the scope of the disclosure or the embodiments disclosed herein. Having now described the present disclosure in detail, the same will be more clearly understood by reference to the following examples, which are included for purposes of illustration only and are not intended to be limiting of the disclosure.

### EXAMPLES

The following examples are non-limiting. They are included to demonstrate various embodiments of the present disclosure and are provided for illustrative purposes only. These examples are not meant to constrain any embodiment disclosed herein to any particular application or theory of operation. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction for Examples 1-6.

The drug development pipeline is a long and rigorous pathway that commonly leads to hundreds of millions of dollars being wasted in pre-clinical drug screening. Costly issues faced during drug development include identifying efficacious therapies and verifying that those compounds will be safe in the clinic. Accordingly, many efforts are being put forth to develop sensitive cell-based pre-clinical screening systems that can identify efficacious and safe compounds prior to initiation in costly and dangerous phase I clinical trials. The advantage of such models is that they can be scaled up to high throughput formats, which enables the screening of tens of thousands of compounds simultaneously. One major area of focus is on the development of liver models since many new therapies are being developed to target liver disease while hepatotoxicity remains a major cause for post market withdraw of compounds.

Owing to species-specific differences in drug metabolism enzymes, human relevant systems are needed to properly model human drug metabolism/toxicity/efficacy. Additionally, to model human diseases, such as viral infection and fatty liver disease, human based systems will likely be necessary since animal models are not susceptible to infection, as is the case with hepatitis C, and disease development is significantly different in humans. Cell based systems, ranging from simple 2D cultures up to complex bioprinted 3D tissues, have gained the most interest for these areas of drug development. Regardless of culture setup, primary human hepatocytes (PHHs) remain the gold standard for the pharmaceutical industry since they retain the entire set of drug metabolism enzymes necessary for processing candidate compounds and they accurately predict human drug metabolism/toxicity. The drawback to using PHHs is that they rapidly, 24-72 hours, lose their liver phenotype in vitro. The liver tissue engineering field has evolved around this issue and consequently, methods have been developed to prevent the premature decline of PHHs. Unfortunately, even with the major advances that have been made, hepatocyte in vitro functional lifetime seems to be limited to 4-6 weeks, while the in vivo lifetime is expected to be >1 year.

One area that has lacked substantial improvement over the years is the formulation of cell culture medium. Current culture systems either oversupply nutrients and hormones to cells while using animal based products, such as serum, or undershoot the vast complexity of the soluble factors found in the body using chemically defined medium. Once the ideal liver culture system is designed, these soluble cues will be major determining factors for cell function and lifetime. Additionally, for regenerative medicine purposes, all human or synthetic based cell culture medium supplements will be necessary to prevent non-human component induced immune reactions, and the transmission of infectious agents.

Recently it has been shown that physiologic concentrations of glucose and bile acid supplementation to culture medium can prevent an insulin resistant and inflamed state from developing in hepatocytes, respectively. Inspired by this work and the remaining issues with liver cell culture, a xeno-free cell culture medium with physiologically relevant concentrations of insulin and glucose was developed to address how a more physiologically relevant medium might enhance hepatocyte lifetime and functionality. An already established long-term hepatocyte culture system was utilized to address how this xeno-free cell culture medium may provide any significant advantage over similar animal based components. In this culture system, hepatocytes are micropatterned onto collagen domains to facilitate homotypic, self-self, interactions and then surrounded by supportive 3T3-J2 murine embryonic fibroblasts, which enables important heterotypic interactions. These micropatterned co-cultures (MPCCs) have a lifetime of 4-6 weeks and are normally cultivated in animal based products, which provides a system to address how a xeno-free culture medium may further improve cultures that already have a significantly high level of function and lifetime. Over 4-10 weeks, how this new medium formulation affects hepatocyte morphology, lifetime, functions, transporter formation and insulin sensitivity was assessed. Additionally, it was verified that this system could still be used to identify hepatotoxic and non-toxic compounds as well as drug-drug interactions.

### Example 1. Optimizing Culture Medium to Enable Proper Comparison of Physiologically Relevant Medium to Traditional Medium

Traditionally, hepatocyte maintenance medium utilizes bovine serum and excessive amount of glucose as well as insulin. Bovine serum could potentially have proteins which negatively affect hepatocyte insulin pathways on human hepatocytes while it could also contain pathogenic proteins or cause an immune response in humans if used for regenerative medicine purposes. Additionally, high amounts of glucose and insulin are major contributors to fatty liver disease and this is recapitulated in the MPCC system. These abnormalities in culture medium inspired the development of a more physiologic medium that did not contain any animal products, xeno-free medium. Specifically, a medium was formulated that had physiologic glucose (˜5 mM), human serum (in place of bovine serum) and physiologic levels of insulin (0.5-1 pM versus the traditional 1 μM).

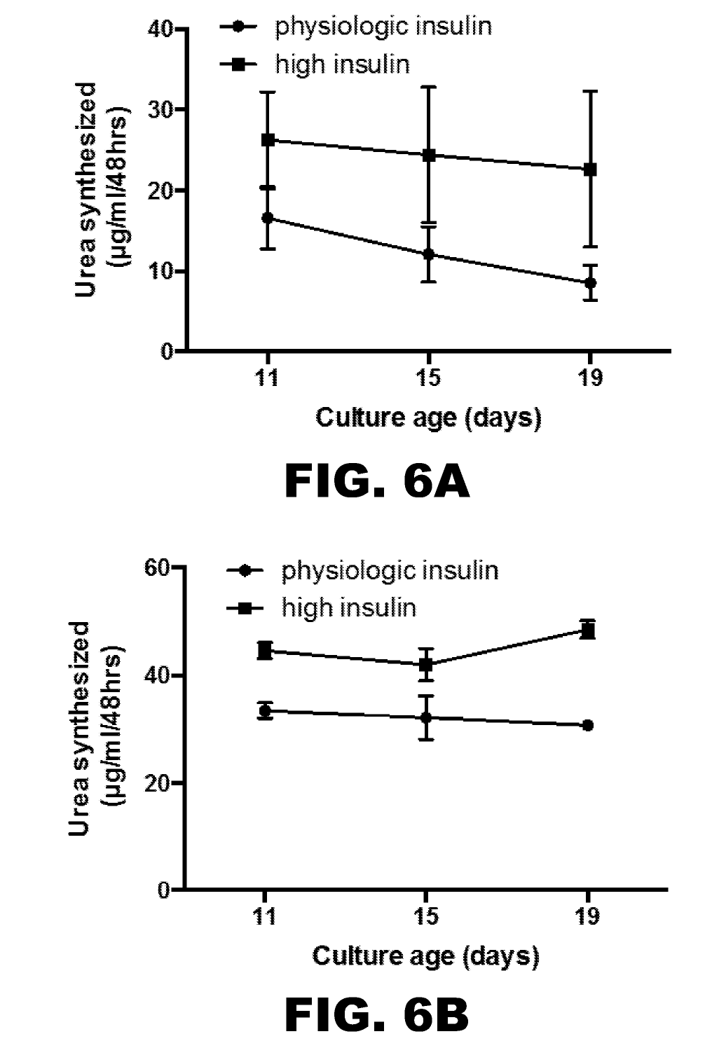

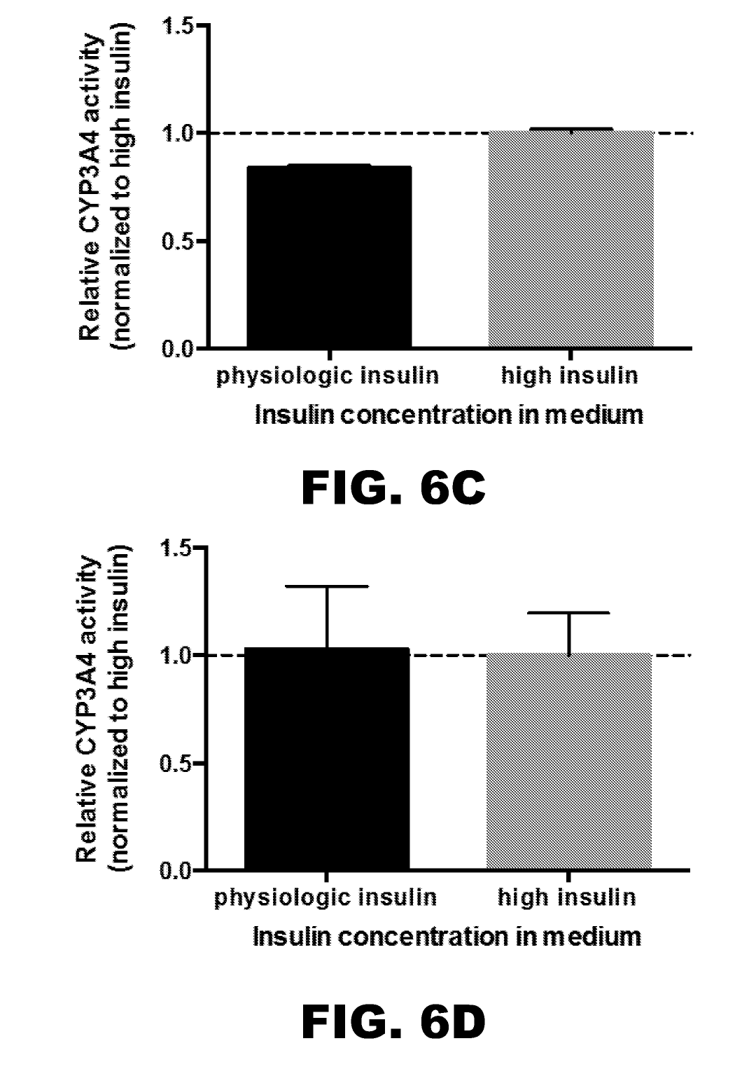

Since glucose levels can be reduced in medium without adverse effects on MPCC performance, experiments focused on how changing insulin levels and serum type affected hepatocyte function and longevity. Surprisingly, it was found that reducing insulin to physiologic levels in MPCCs cultured with bovine serum led to declining hepatocyte urea production and lower CYP3A4 activity, whereas lowering insulin in MPCCs cultured with human serum maintained stable hepatocyte urea production and CYP3A4 activity after 3 weeks in culture (FIG. 6). These results suggested that human serum enables culturing MPCCs in physiologic concentrations of insulin, whereas MPCCs cultured in bovine serum decline without excessive insulin supplemented in culture medium. Consequently, the remainder of these studies were carried out using the traditional amount of insulin (1 μM) in cultures with bovine serum containing medium, while cultures carried out in medium with human serum had physiologic (500 pM) levels of insulin.

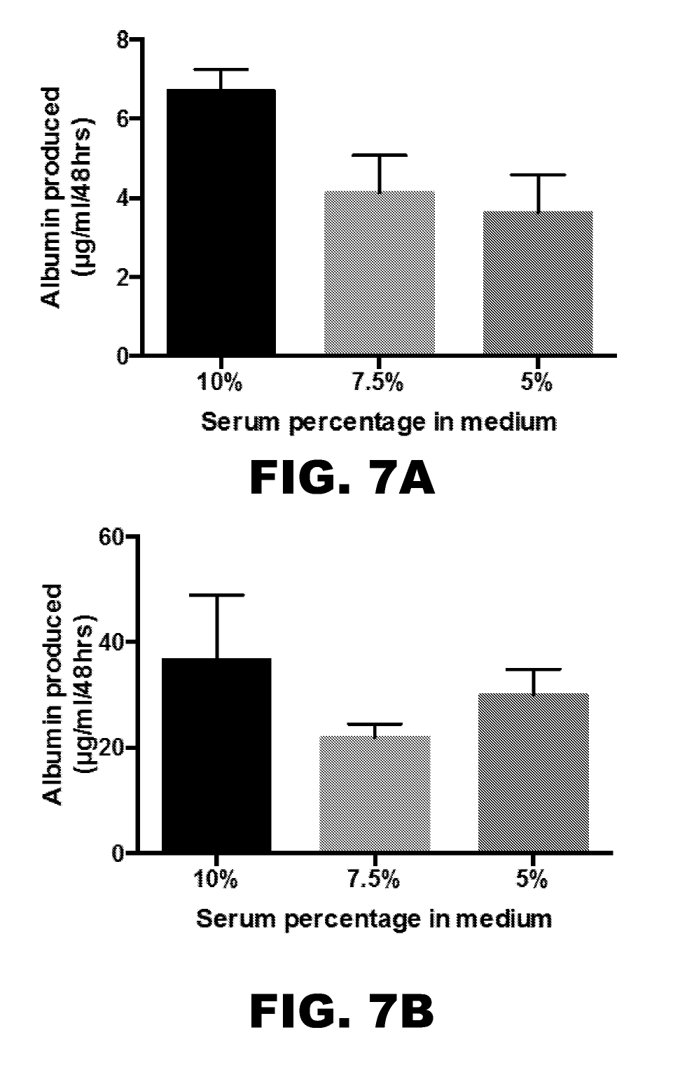

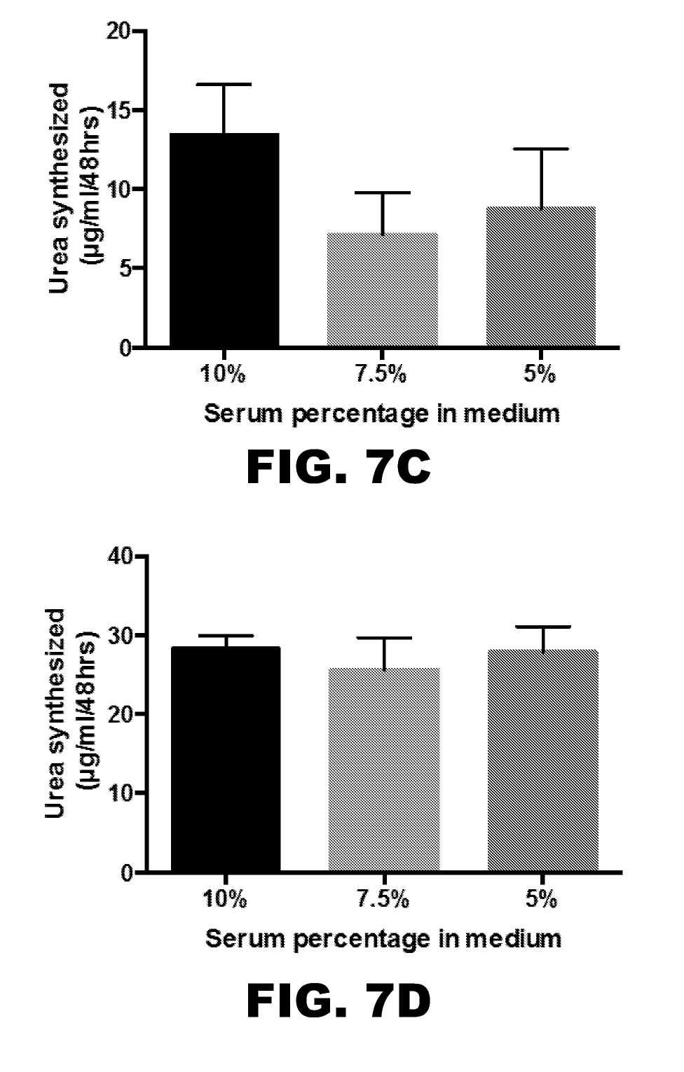

To further optimize the medium formulation, MPCCs were cultured in different percentages of serum and the effects of serum concentration on hepatocyte albumin production and urea synthesis were assessed. Within the range tested, 5-10% serum containing medium, no significant change in hepatocyte function with different amounts of human serum was found, while we did find that 10% bovine serum seemed to be the optimal percentage of serum for MPCC albumin production (FIG. 7). Therefore, subsequent studies were carried out using 10% human and 10% bovine serum. Since fibroblasts enable hepatocyte functions in MPCCs and a proper comparison between physiologic medium and the traditional medium was desired, fibroblasts were growth arrested using mitomycin C prior to incorporation into MPCCs (FIG. 1). Growth arrested fibroblasts can still support hepatocytes while their growth and numbers should be consistent with different medium supplements.

### Example 2. Physiologically Relevant Medium Prolongs the Lifetime of Hepatocytes in Micro Patterned Co-Cultures

Once the optimal medium formulations were identified, long term studies (10 weeks) were carried out to assess the possibility of culturing hepatocytes in a xeno-free and physiologically relevant medium, where hepatocyte colony morphology, and functions over time was assessed. Surprisingly, physiologically relevant medium was not only compatible with MPCCs, but hepatocyte colony morphology, as assessed by phase contrast imaging, showed dramatic stabilization of hepatocytes compared to traditional medium (FIG. 1). Specifically, hepatocyte morphology and island integrity was diminished by 6 weeks in traditional medium, whereas hepatocyte morphology was greatly retained up to 10 weeks using physiologically relevant medium.

### Example 3. Hepatocyte Functional Lifetime is Significantly Longer in Physiologic Medium

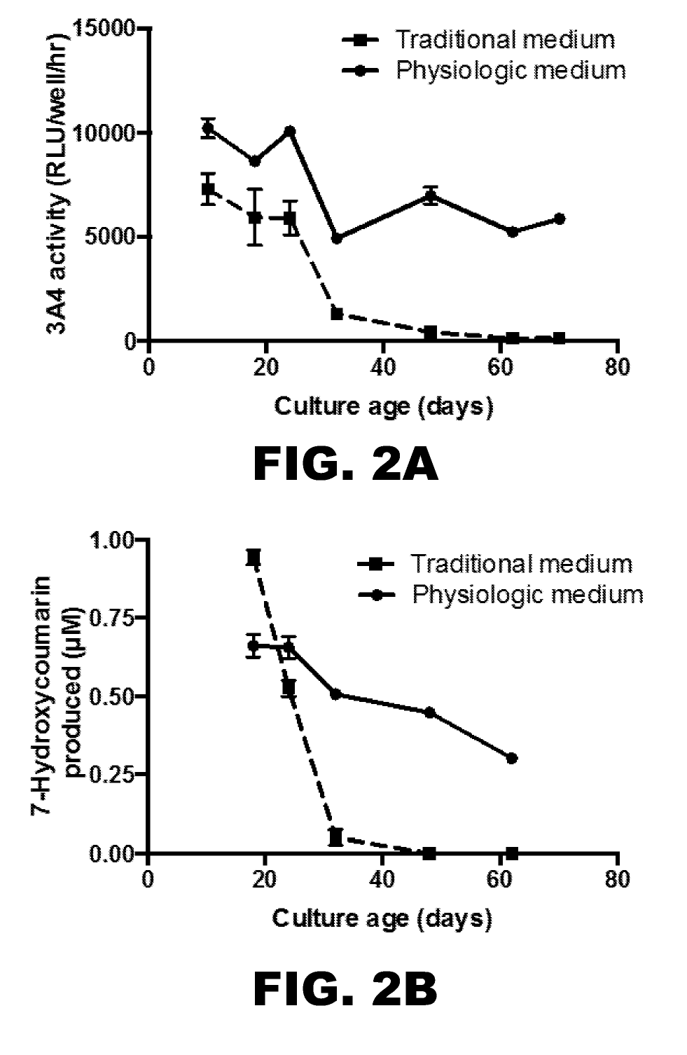

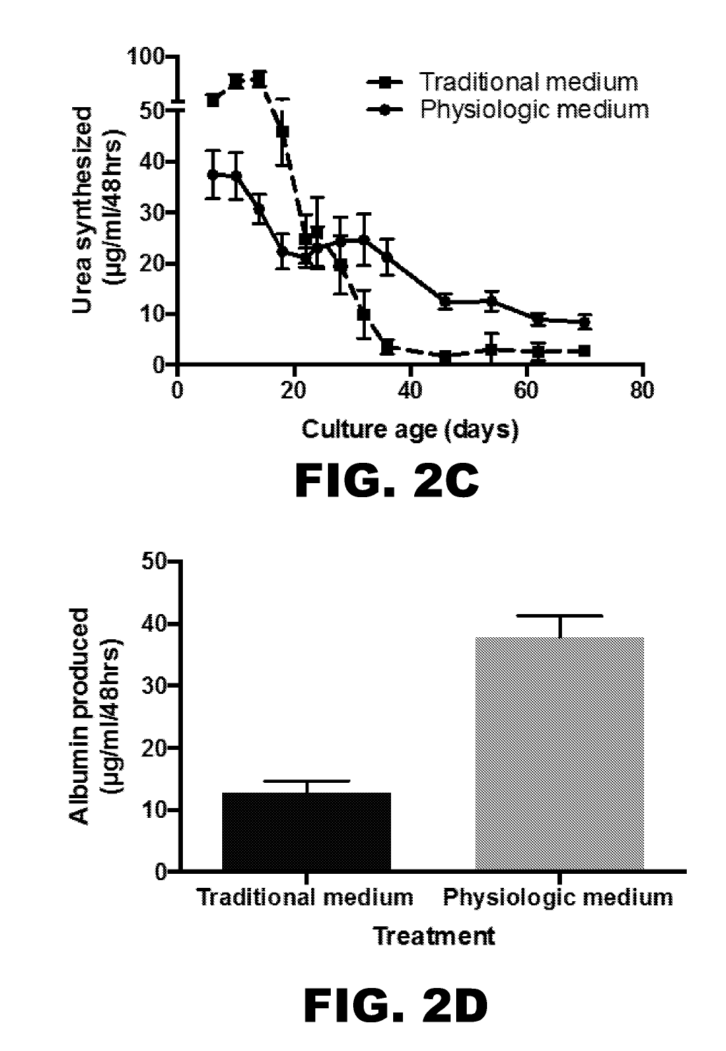

Along with hepatocyte morphology, hepatocyte functions were also maintained in physiologic medium. Specifically, when CYP3A4 activity was measured in MPCCs over time, it was found that traditional medium enzyme activity was ˜1% of 1 week levels, while physiologic medium MPCC enzyme activity was ˜60% of 1 week levels (FIG. 2). CYP2A6 enzyme activity was also assessed for 8 weeks, and traditional medium CYP2A6 activity was completely absent by 62 days in culture, while physiologically relevant medium CYP2A6 levels were still 45% of 1 week levels.

Urea synthesis was also maintained in physiologic medium at 57% and 23% of 1 week levels at 5 and 10 weeks in culture, respectively. Alternatively, urea synthesis of MPCCs in traditional medium was ˜7% and 5% of 1 week levels by 5 weeks and 10 weeks in culture, respectively. Importantly, albumin production was ˜3 fold higher at 3 weeks of culture in physiologic medium relative to traditional medium. These results suggest that physiologic medium supports a high level of hepatocyte function in MPCCs while also prolonging the lifetime of hepatocytes in culture.

### Example 4. Polarized Hepatocyte Transporters Remain Intact in Physiologic Medium

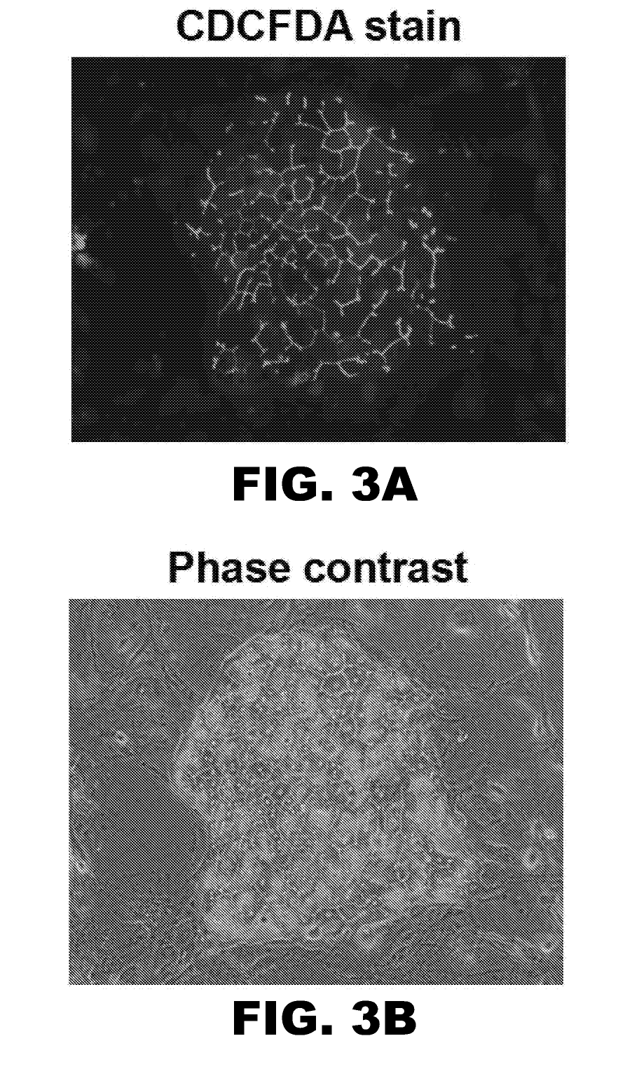

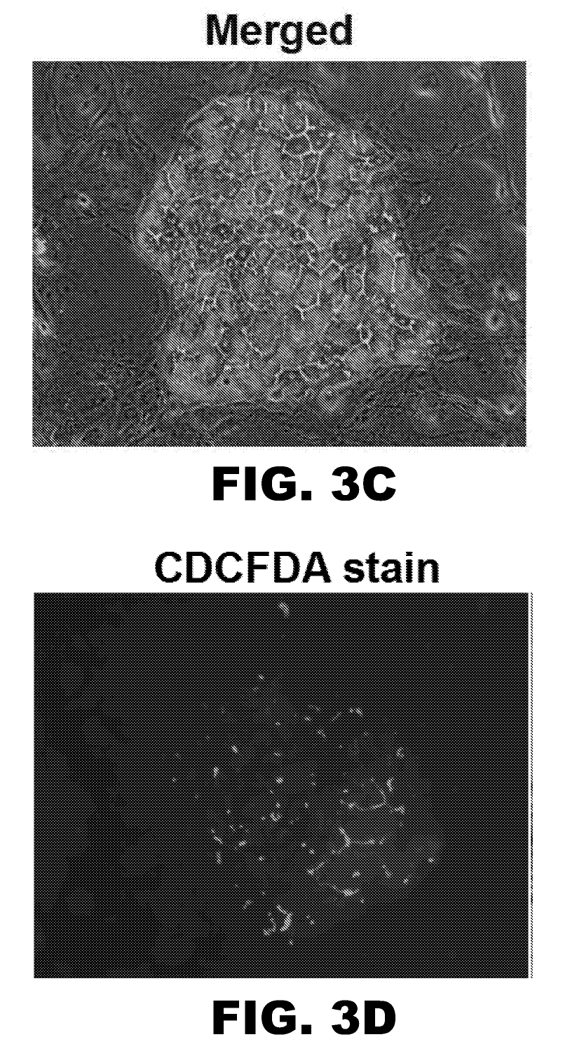

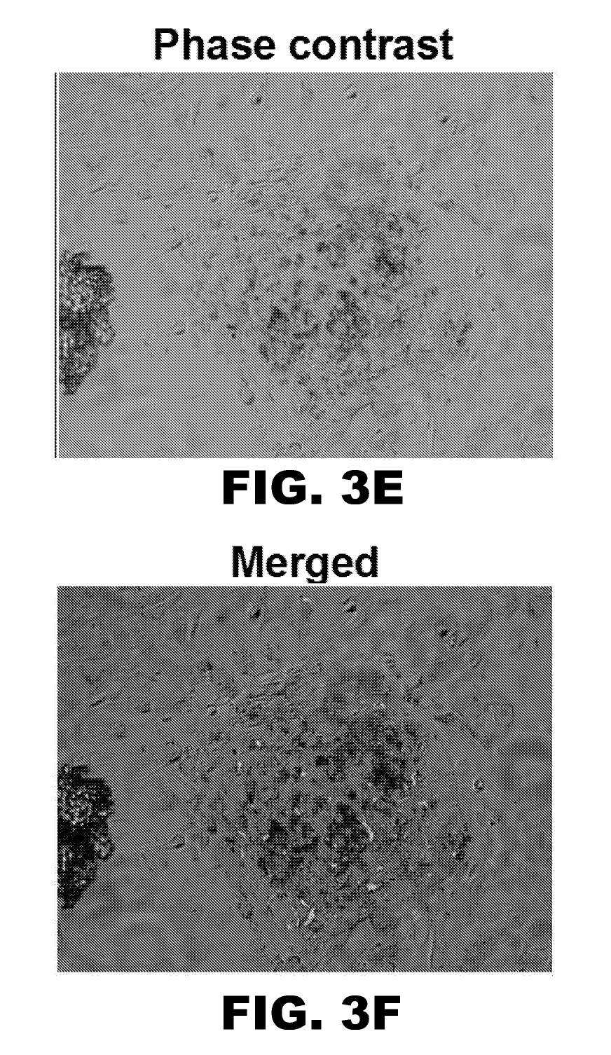

Since hepatocyte islands had sustained integrity in physiologic medium, it was hypothesized that hepatocyte islands would have greater levels of transporter function, which requires a high degree of homotypic hepatocyte-hepatocyte interactions. To probe transporter function, a fluorescent dye was used which is selectively transported through the hepatospecific multidrug resistant-like proteins 2 and 3 (MRP2, −3). At early time points (˜2 weeks, data not shown) hepatocytes had significant transporters in all conditions, while after 4 weeks there was a significant loss of transporters in hepatocyte islands cultured in traditional medium (FIG. 3). Importantly, hepatocyte transporters were mostly retained in hepatocyte islands cultured in physiologic medium. This loss or retention of transporters correlated with the retention of island integrity, as shown by corresponding phase contrast images. These results suggest that physiologic medium retains hepatocyte polarity and transporters over at least 4 weeks in vitro.

### Example 5. Hepatocyte Insulin Sensitivity is Retained in Physiologically Relevant Medium

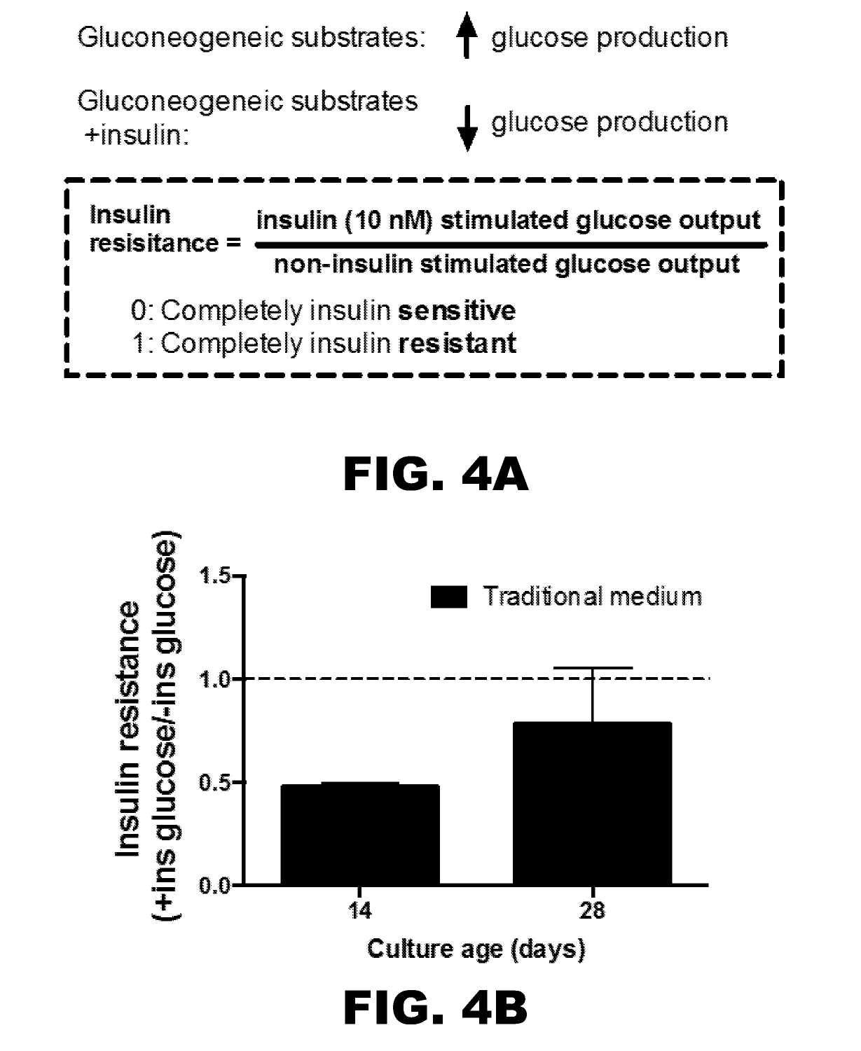

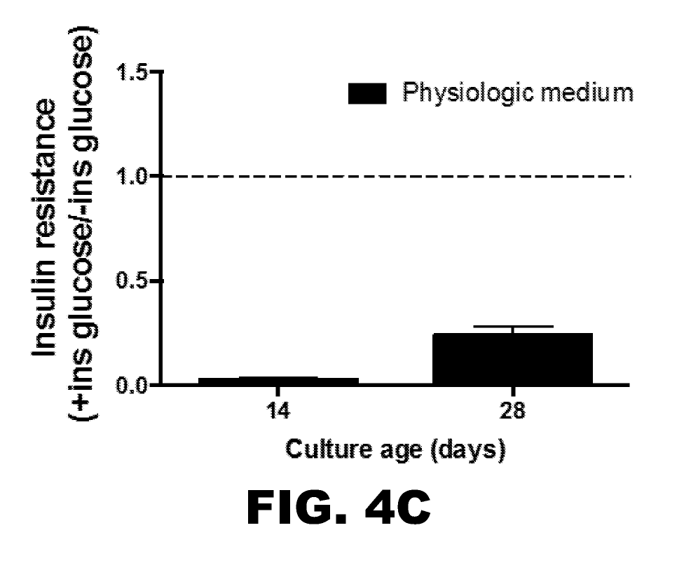

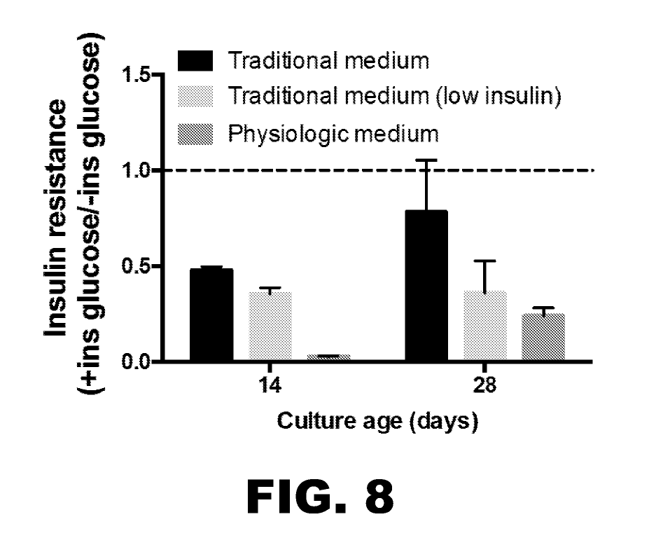

One critical function of the liver is to maintain glucose levels in the blood by responding to hormones secreted from the pancreas and to a lesser extent by factors secreted from adipose tissue. This function of liver cells is almost always overlooked in in vitro liver models, although it is a key determinant of liver health. Additionally, bovine factors, such as fetuins, and high amounts of insulin could contribute to insulin resistance in vitro. Therefore, MPCC insulin resistance after treatment with traditional or physiologic medium over 4 weeks was assessed. Insulin resistance is calculated by dividing the insulin-stimulated glucose output from cultures by the non-insulin stimulated glucose output over 8 hours of glucose production, where completely insulin resistant cultures have a value of 1 and completely insulin sensitive cultures have a value of 0 (FIG. 4). It was found that hepatocytes in traditional medium were ˜50% insulin resistant after 2 weeks and completely insulin resistant after 4 weeks of culturing in traditional medium. Alternatively, hepatocytes cultured in physiologic medium were almost completely insulin sensitive, 2.5% insulin resistant, after 2 weeks of culture, and 23% insulin resistant after 4 weeks of culture. This increased rate of insulin resistance was likely from bovine serum factors, since even hepatocytes cultured in physiologic insulin (˜500 pM) developed insulin resistance faster than cultures in human serum (FIG. 8). These results suggest that physiologic medium retains insulin sensitivity in hepatocytes longer than traditional medium.

### Example 6. Physiologically Relevant Medium Allows for Sensitive and Specific Hepatotoxicity Screening as Well as Drug-Drug Interactions

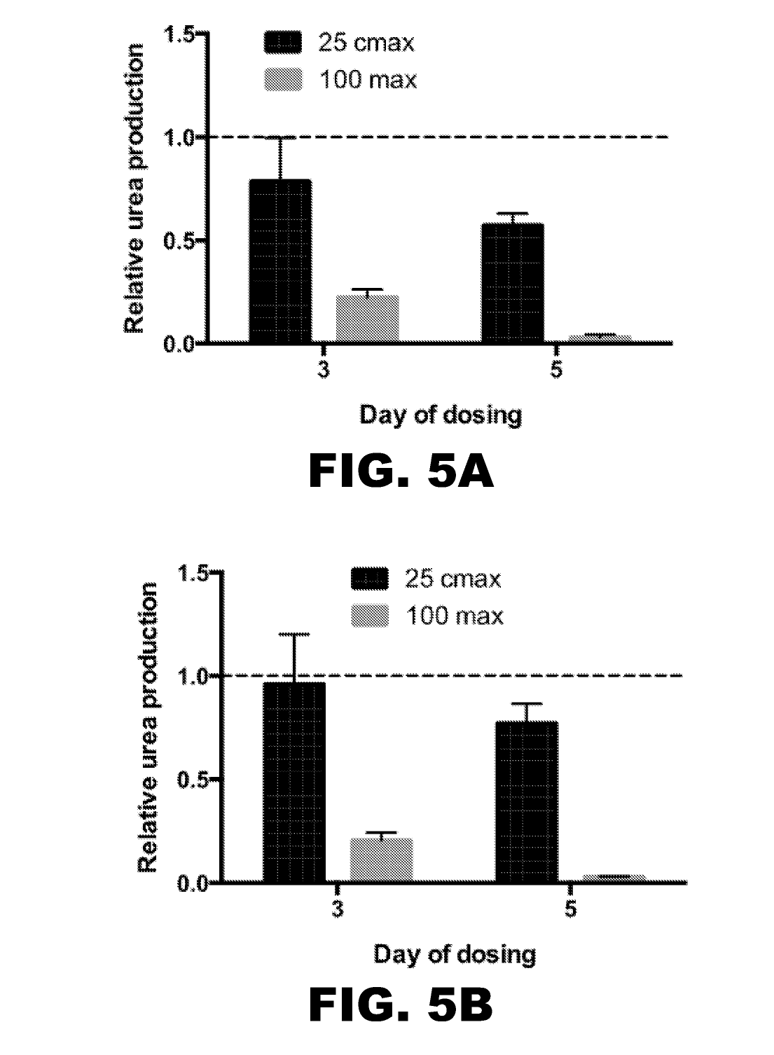

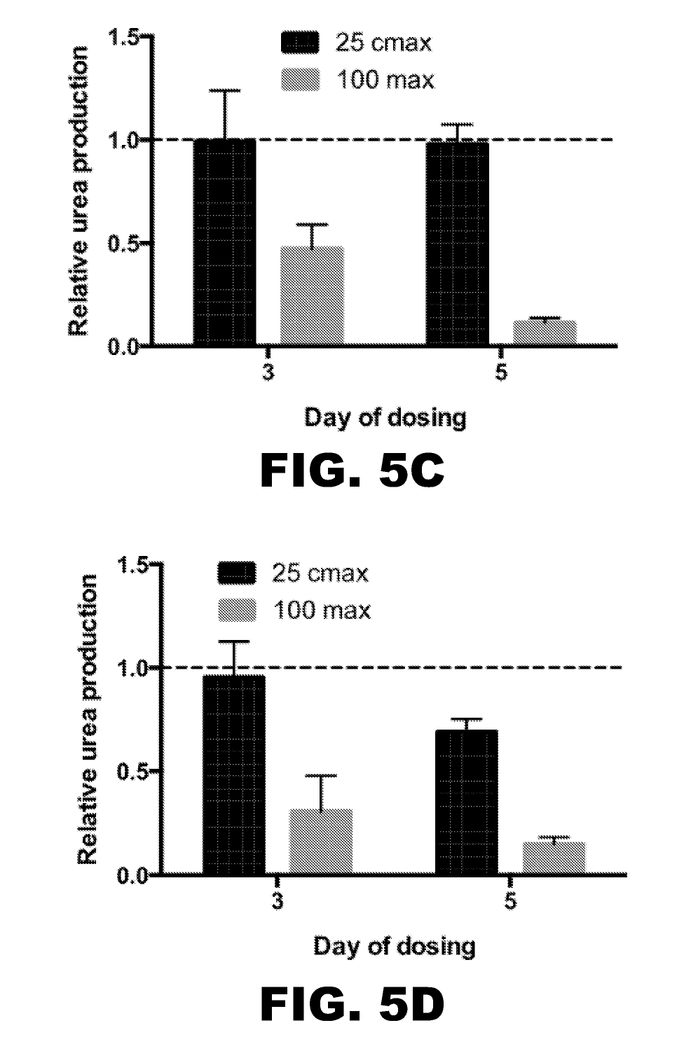

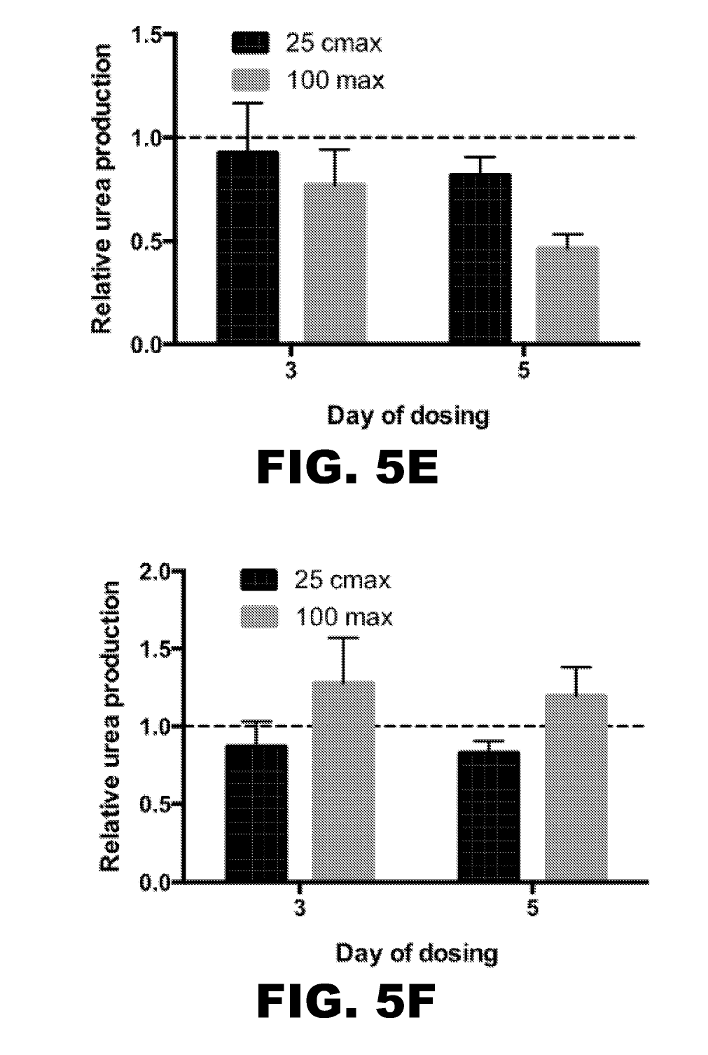

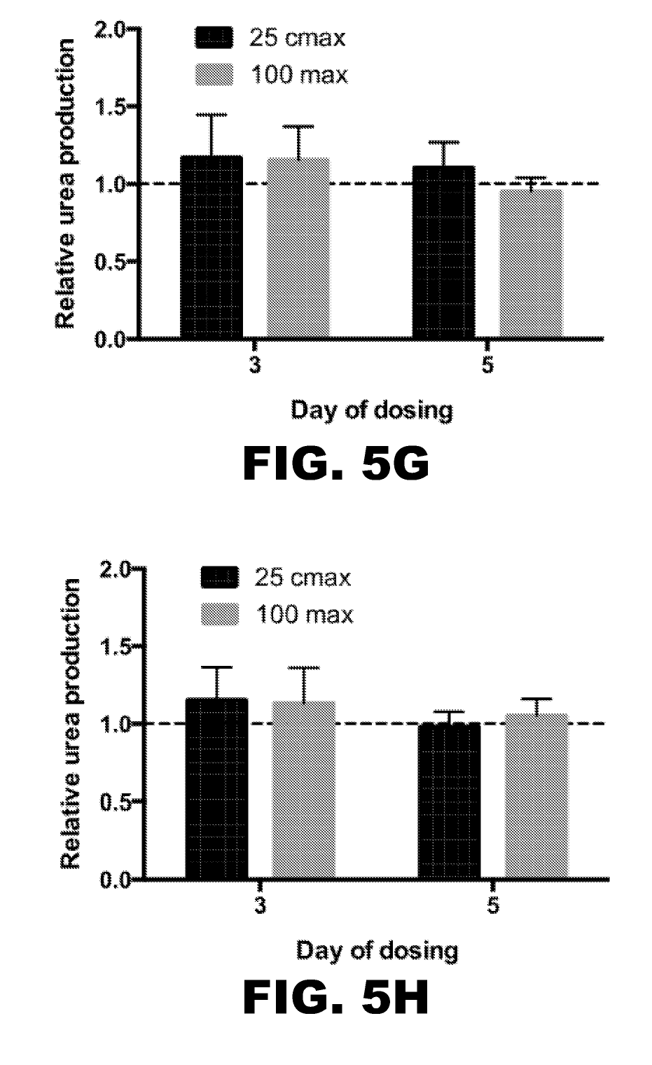

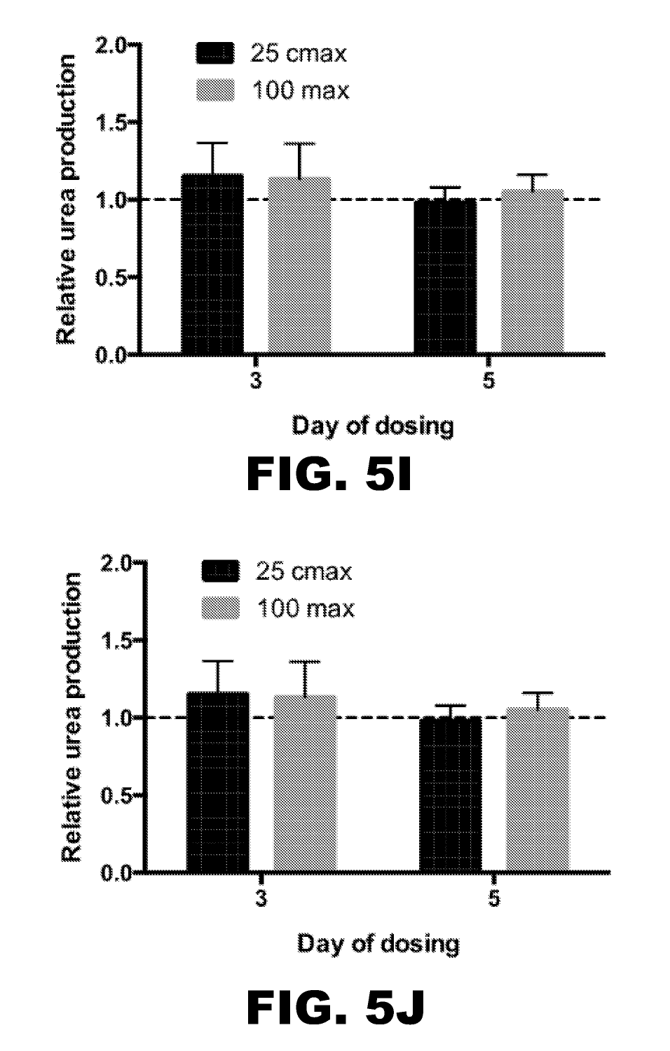

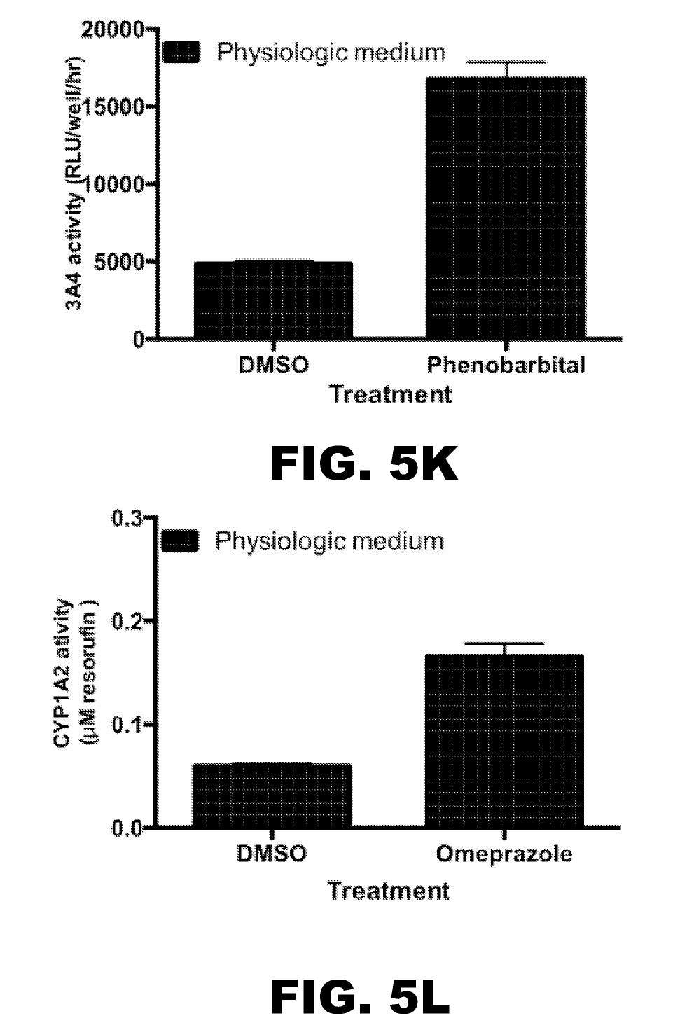

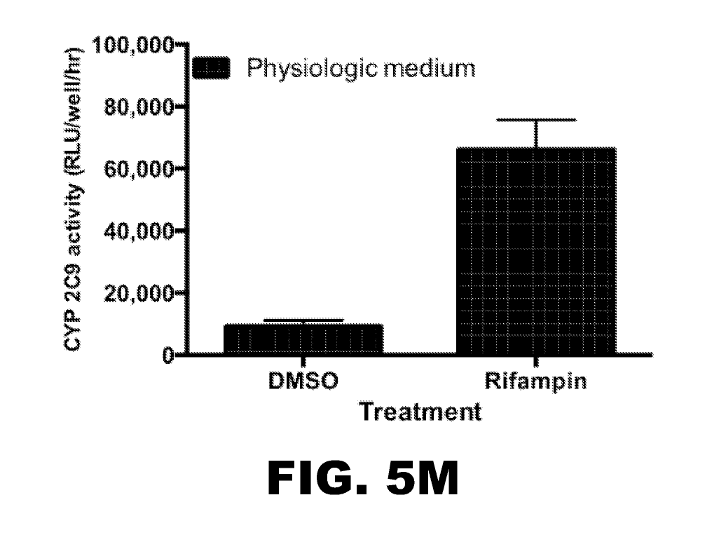

Since these changes in medium formulation led to increased longevity, function and hormone responses, it was necessary to ensure that these cultures could also accurately predict hepatotoxins and drug-drug interactions. Therefore, a drug screen with cultures after they were subjected to physiologic medium for 10 days was carried out (FIG. 5). Specifically, cultures were treated with 5 known hepatotoxins, and 5 non-toxins 2 times over a period of 5 days with increasing concentrations of Cₘₐₓ (25*Cₘₐₓ, and 100*Cₘₐₓ), the average maximal drug concentration observed in patients after administering the drug. The compounds tested have previously been identified as hepatotoxins or non-toxins in comprehensive hepatotoxicity studies. Drug treatments were carried out in serum free medium to prevent potential drug-protein interactions. Compounds were categorized as toxic if urea synthesis in MPCC supernatants, which has recently been shown to be a more sensitive marker of liver toxicity than ATP, dropped below 50% of the vehicle (DMSO) treated control. After 2 treatments with toxins, MPCCs cultured in physiologic medium correctly identified all 5 hepatotoxins (diclofenac, troglitazone, clozapine, amiodarone and piroxicam) as toxic, as shown by a concentration and time dependent decrease in urea synthesis (FIG. 5). Calculated TC₅₀ values, the interpolated concentration of the drug where urea synthesis drops below 50% of the vehicle control, for the various compounds were 851 μM, 1555 μM, 191 μM, 340 μM and 2901 μM for diclofenac, troglitazone, clozapine, amiodarone and piroxicam, respectively (Table 1). Importantly, MPCCs in physiologic medium also correctly categorized all 5 non-toxins (aspirin, dexamethasone, rosiglitazone, prednisone and miconazole), as shown by no significant loss in urea synthesis over time with increasing concentration of compound (FIG. 5). These results suggest that hepatocytes cultured in physiologic medium retain the ability to correctly identify potential hepatotoxins and non-toxic compounds.

TABLE 1
Calculated TC₅₀ values for cultures in physiologic medium.

| | Hepatotoxicity screening | | |
|---|---|---|---|
| Compound tested | | Calculated TC₅₀ | Cₘₐₓ |

| | | | |
|---|---|---|---|
| Diclofenac | 851 | μM | 8.023 μM |
| Troglitazone | 1555 | μM | 6.387 μM |
| Clozapine | 191 | μM | 0.951 μM |
| Amiodarone | 340 | μM | 0.806 μM |
| Piroxicam | 2901 | μM | 5.135 μM |

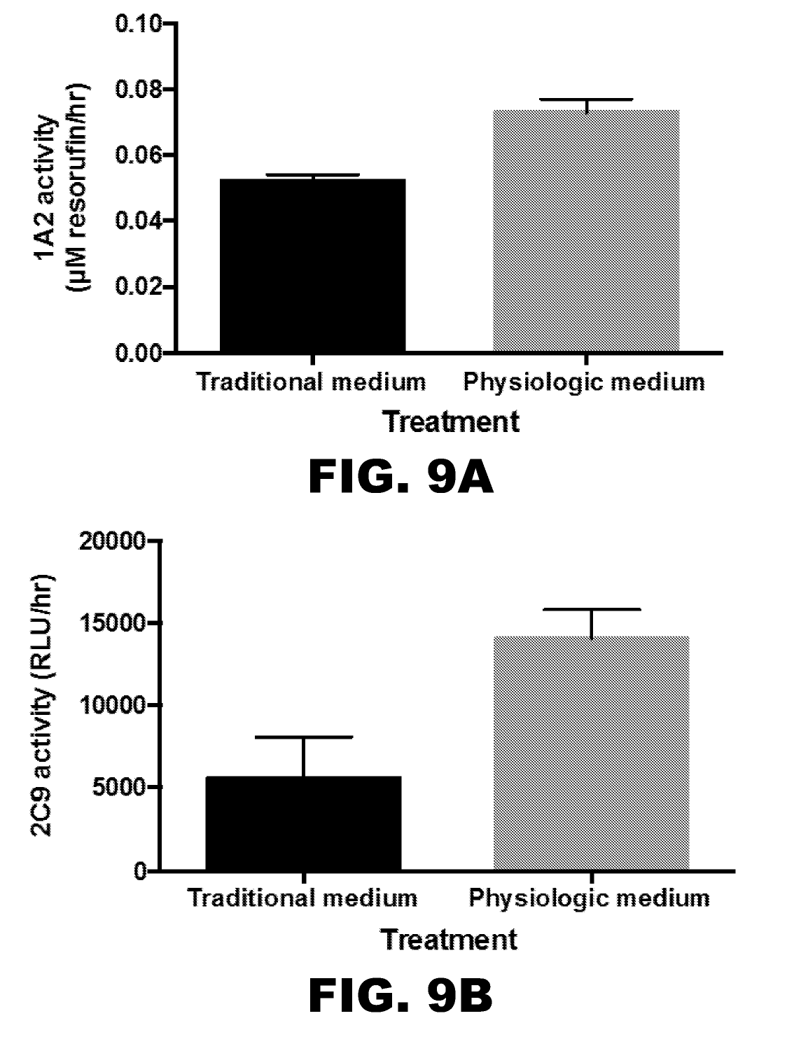

To assess the potential of MPCCs in physiologic medium to predict drug-drug interactions, cultures were treated with known enzyme inducers and their enzyme activity was quantified. Specifically, to induce CYP3A4, CYP1A2 and CYP2C9, MPCCs were treated twice with phenobarbital, omeprazole or rifampin, respectively, over 4 days in serum free medium. Cultures were treated with the respective vehicle control, water or DMSO, as a non-induced control. Induction was carried out after 10 days of culture in physiologic medium. MPCCs in physiologic medium showed a 3.5 (±0.25) fold induction of CYP3A4 activity, 2.75 (±0.24) fold induction of CYP1A2 and a 7.2 (±1.8) fold induction of CYP2C9 activity (FIG. 5). Slightly higher levels of basal CYP1A2 and CYP2C9 activity was found in MPCCs after 3 weeks of culture in physiologic medium compared to traditional medium (FIG. 9). These results suggest that MPCCs cultured in physiologic medium retain the ability to identify potential enzyme induction and drug-drug interactions.

### Discussion for Examples 1-6.

Primary human hepatocytes are the gold standard for pharmaceutical drug screening and also the development of bio-artificial livers and implantable constructs for regenerative medicine applications. Since hepatocytes are inherently difficult to maintain outside of the body this poses significant hurdles for drug screening and regenerative medicine applications and any advances in maintaining hepatocytes outside the body should benefit these areas. One area of optimization that can be broadly applicable across these inherently different research fields is advancements in the medium by which nutrients and signaling proteins are supplied to cell and tissue cultures. A xeno-free culture medium was developed that better mimics the physiologic concentrations of glucose and insulin in vivo while maintaining the complexity of human blood profile by using pooled human serum. Surprisingly, it was found that the physiologically relevant medium formulation developed could not only support normal hepatocyte functions enabled by micropatterned co-cultures (MPCCs), but that it significantly prolonged the lifetime and functions of hepatocytes in this co-culturing system without losing its utility for drug screening and glucose metabolism studies.

The use of human serum, more than any other modification made, is what enabled the long lifetime and functions of hepatocytes, as hepatocytes were sustained for extended periods of time over cultures with bovine serum even with abnormal levels of insulin and glucose. Although, the overall goal was to develop a more physiologically relevant medium that would be useful for a variety of applications, including disease modeling, the culture medium was further modified to have the ideal amount of serum and insulin. Surprisingly, it was found that by using 10% human serum in the medium, the amount of insulin supplemented in medium could be lowered down to a physiologically relevant concentration, ˜500 pM, while this amount of insulin supplementation in medium containing bovine serum as a base led to declining hepatocyte functions. Using this optimized xeno-free medium, hepatocyte functions, such as drug metabolism enzyme activity, urea synthesis and albumin production, were retained longer than traditional medium. Serum is a complex mixture of proteins, lipids and biomolecules, and how individual components of serum may prolong the lifetime of hepatocytes in vitro is being assessed.

When human serum is supplemented for bovine serum there is a substantial change in hepatocyte morphology and this is reflected with more cuboidal cells, distinctive hepatocyte boarders (between surrounding fibroblasts and hepatocyte islands), and extensive bile canaliculi. This was confirmed with hepatocyte transporter specific dye export, where the fluorescent dye accumulated in the canaliculi between hepatocytes. The hepatocyte colonies in MPCCs cultured in physiologic medium are sustained for an unprecedented 10 weeks in vitro. Over the last 2 weeks of culture, the island geometry begins to morph into random shapes, rather than the initial circles they were patterned in, while the loss of cells seems to be much slower than colonies in bovine serum.

One major benefit of the MPCC is that it greatly retains the ability of hepatocyte to respond to insulin. Unfortunately, insulin signaling studies must be carried out within the first 2 weeks of culture or else hepatocytes lose the ability to respond to insulin, which is likely due bovine serum factors, such as fetuins, and overstimulation with excessive amounts of insulin in traditional medium. Accordingly, hepatocytes in physiologic medium, which has low levels of insulin, greatly retained the ability to respond to insulin even after 4 weeks in vitro, while cultures in traditional medium were completely insulin resistant by this time. Another possibility for the retained lifetime of hepatocytes in physiologic medium, is that bovine serum has components, such as fetuins, which may cause the decreased insulin signaling (insulin resistance), which could lead to premature cell death since insulin signaling is necessary for cell survival. Along with this theory, hepatocyte insulin resistance occurred at a much higher rate in hepatocytes cultured with bovine serum, compared to cultures in human serum. Additionally, even cultures in bovine serum with low insulin, which have decreased hepatic functions, also developed insulin resistance at a faster rate than cultures in human serum. This has major implications for diabetes and insulin signaling research since MPCCs, or possibly other liver cell culture platforms, could be maintained in physiologic medium while also supplementing various factors that are implicated in insulin resistance development to ultimately identify mechanisms behind disease progression in a human relevant system.

Lastly, the most widely used application of the MPCC platform is drug toxicity screening and it was found that physiologic medium did not prevent the platform from correctly identifying potential hepatotoxins and drug-drug interactions. Since hepatocyte functions are sustained for extended periods of time in this physiologic medium, it is expected to also obtain highly sensitive drug screening results after prolonged periods of culture. This will be especially important for liver disease modeling applications where disease development takes extended periods of time to develop, such as non-alcoholic steatohepatitis (NASH) and fibrosis.

This medium formulation should also aid regenerative medicine efforts for multiple reasons. The most common methods of engineering liver tissue constructs to potentially replace failing livers, is to fabricate and stabilize the tissue in vitro, and then implant the construct. This optimized medium should greatly enhance the stabilization of liver cells prior to implantation and also prevent them from developing disease phenotypes prior to implantation. Additionally, for the FDA to approve implantable engineered tissue constructs in the future, these tissues will need to be created using good manufacturing practices (GMP), which prevents the use of xeno-based factors. This work provides a substantial advance for hepatocyte culturing while considering this aspect.

### Methods for Examples 1-6.

Cell Culture and MPCC Fabrication.

MPCC fabrication was carried out as previously described. Growth arresting fibroblast cultures and seeding into MPCCs was carried out as previously described. To account for potential differences in fibroblast growth and spreading between physiologic medium and traditional medium, cultures were first created and maintained in traditional medium for 4 days and then switched their respective medium.

Physiologic Medium.

Base medium: Dulbecco's Modified Eagle's Medium without glucose or Williams E Medium without glucose
1-4 mM L-glutamine
1-25 mM Glucose
0.1-100 nM Insulin
0-6.35 μg/ml Transferrin
0-6.25 ng/ml Selenium
0-500 nM Dexamethasone
0-2 nM Glucagon
0-15 mM HEPES ((4-(2-hydroxyethyl)-1-piperazineethanesulfonic acid)
0-100% Human serum.

Traditional Medium.

Base medium: Dulbecco's Modified Eagle's Medium without glucose
4 mM L-glutamine
0-25 mM Glucose
1000 nM Insulin
6.35 μg/ml Transferrin
6.25 ng/ml Selenium
100 nM Dexamethasone
2 nM Glucagon
0-15 mM HEPES ((4-(2-hydroxyethyl)-1-piperazineethanesulfonic acid)
0-20% serum from bovine sources of different age groups, including fetal, calf and adult.

Biochemical and Enzyme Activity Assays.

Urea synthesis and glucose production was quantified using previously described methods. Since human serum contains significant amounts of human serum, an alternative protocol to quantify MPCC albumin production was used. Specifically, after 2 and 3 weeks of culture, cultures were washed 3 times with 1×PBS, and then incubated for 24 hours in serum free medium. Albumin was then measured in this medium using a sandwich ELISA method. To account for potential residual albumin in cell culture medium, any differences in albumin production between cultures incubated in 5%, 7.5% and 10% serum were examined. No significant differences in albumin production between these cultures was observed, which suggested that most residual albumin had been successfully removed with the PBS washing steps prior to the 24 incubation period. Enzyme activity was assessed using the methods previously described.

Imaging and Transporter Visualization.

Bile canaliculi (CDCFDA) and phase contrast imaging was carried out as previously described. Importantly, bile canaliculi imaging was carried out after 4 weeks of culture in these studies.

Insulin Resistance Assay.

To assess the insulin sensitivity of these cultures, cultures were first cultured in hormone free, serum containing medium for 24 hours, and then washed 3 times with 1×PBS to remove residual glucose. Cultures were then incubated in glucose free medium containing 4 mM L-glutamine, 1% penicillin/streptomycin, 1.5% HEPES buffer, 20 mM lactate and 2 mM pyruvate to and +/−10 nM insulin. Insulin resistance was calculated by dividing the insulin-stimulated glucose output by the basal level of glucose output.

Drug Toxicity and Enzyme Induction Studies.

Drug toxicity and enzyme induction were assessed as previously described. Importantly, in these studies, drug toxicity and enzyme induction were carried out after 2 weeks of culture, and the level of these metrics was not directly compared to traditional medium, since these MPCC utilities have clearly been demonstrated in previous publications (Khetani and Bhatia, Nat Biotechnol 2007; 26(1): 120 and Khetani et al., Toxicological Sciences 2013; 132(1): 107), the disclosures of which are hereby incorporated by reference in their entirety.

Statistical Analyses.

Each experiment was carried out in 2 or more wells for each condition. Two to three cryopreserved PHH donors were used to confirm observed trends. Microsoft Excel and GraphPad Prism 5.0 (La Jolla, Calif.) were used for data analysis and plotting data. Error bars on average values represent standard deviation (SD) across wells. Statistical significance of the data was determined using the average and SD across wells in representative experiments using the Student's t-test or one-way ANOVA with Dunnett's multiple comparison tests for post hoc analysis.

### Example 7. A Bioinspired Culture Medium Prolongs the Functional Lifetime of Human Liver Cells in Culture

Given significant differences between animals and humans in liver pathways, in vitro human liver models are essential for preclinical drug screening prior to initiation of clinical trials. Primary human hepatocytes (PHHs) are the “gold standard” for creation of such models and under the appropriate culture conditions, these cells can be kept functional for several weeks in vitro. In particular, micropatterning PHHs onto collagen-coated domains of empirically optimized dimensions and then surrounding them with stromal fibroblasts can maintain major hepatic functions for 3-4 weeks. However, such a lifetime does not allow appropriate modeling of chronic drug outcomes that can take more than 1 month to manifest themselves in PHHs. The culture medium surrounding the cells provides both nutrients and essential factors for maintaining homeostasis. Typically, culture of PHHs is carried out in medium containing sera from bovine sources due to ready availability. However, such a microenvironment does not mimic the composition of human plasma and may lead to premature cell death. Thus, a bioinspired human serum-based culture medium was developed to extend by >2 months PHH functional lifetime.

Soft lithography using polydimethylsiloxane (PDMS) stamps was used to pattern collagen-I into circular domains (500 μm diameter with 1200 μm center-to-center spacing) onto tissue culture polystyrene. PHHs selectively attached to the circular collagen-coated domains and were subsequently surrounded by murine embryonic 3T3-J2 fibroblasts to establish micropatterned co-cultures (MPCCs). Cultures were stabilized in bovine serum for 4 days (to enable fibroblasts to reach confluency), and then transitioned into human serum (10% vol/vol) or left in bovine serum containing medium as a control. MPCC morphology and functions (i.e. urea secretion, albumin production, transporter activity, cytochrome P450 enzyme activities) were measured using well-established assays (Khetani et al., Nat Biotech 2008; 26(1): 120-6, the disclosure of which is hereby incorporated by reference in its entirety). Finally, drugs were tested on MPCCs cultured in the different media types to determine sensitivity and specificity relative to available clinical information.

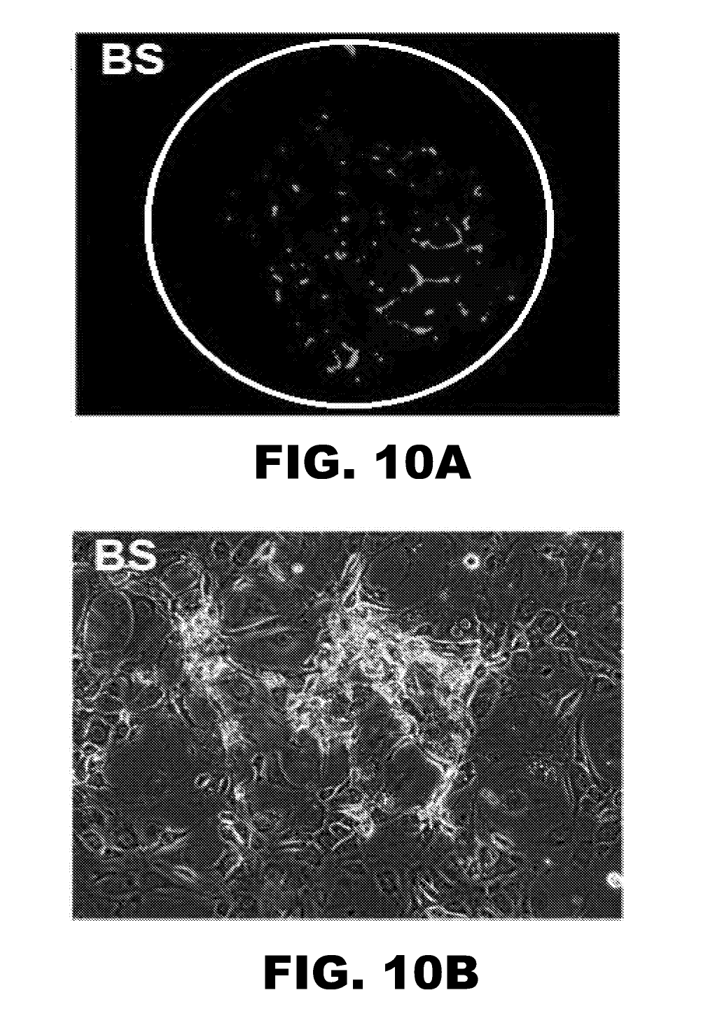

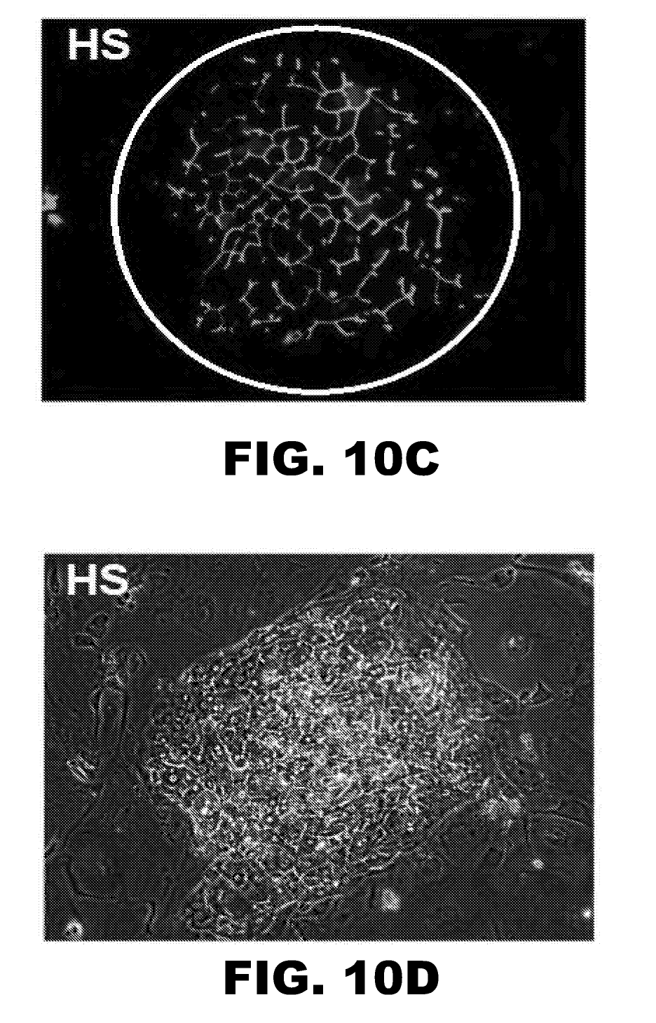

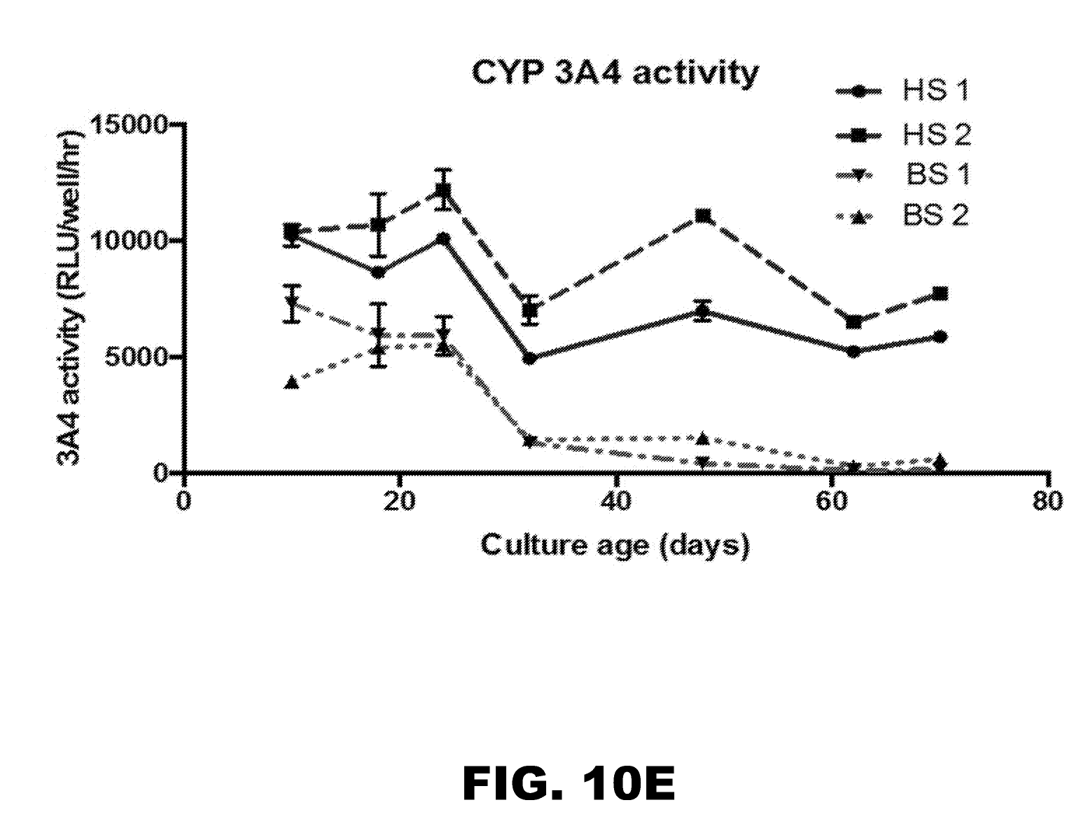

MPCCs maintained in human serum (10% vol/vol) had a substantially longer functional lifetime (˜8-10 weeks) when compared to the traditional bovine serum-based medium (˜3-4 weeks). Specifically, PHHs in the human serum-based medium displayed long-term and higher levels of transporter functions, prototypical hepatic morphology (i.e. polygonal shape, bile canaliculi formation, distinct nuclei/nucleoli), albumin secretion, urea synthesis, and CYP450 activities (i.e. CYP3A4, CYP2A6) relative to the bovine serum control (FIG. 10). Additionally, MPCCs cultured in human serum retained insulin-sensitive reduction in glucose output whereas MPCCs in bovine serum became insulin resistant, a pathological state in type 2 diabetes. Importantly, MPCCs pre-cultured in human serum retained high levels of function and morphology when transitioned into a serum-free medium for drug-mediated CYP450 enzyme induction and toxicity testing, which provides a substantial advancement for pre-clinical drug testing. Drug toxicity studies in MPCCs cultured in human serum yielded toxic/non-toxic classifications that correlated well with available clinical annotation. Different lots of human serum yielded similar improvements in functional longevity, suggesting the reproducibility of this approach as well as an ability to test different patient sera types.

Human serum can improve PHH functional lifetime by at least 4-6 weeks in MPCCs. Use of sera from different patients suggests potential utility in screening for inter-individual variability in drug toxicity outcomes depending on sera composition. The model is now being augmented to include other liver cell types (i.e. macrophages) that can modulate drug toxicity. In the future, it is anticipated that these findings can be broadly applicable to culture systems being developed for regenerative medicine and disease modeling.

1. A method of culturing a population of human hepatocytes in vitro, comprising co-culturing the population of human hepatocytes with at least one non-human, non-parenchymal cell population wherein the composition is incubated with a culture medium comprising human serum obtained from at least one donor.

2. The method according to claim 1, wherein the culture medium comprises from about 5% to about 10% vol/vol human serum.

3. The method according to claim 1, wherein the culture medium further comprises about 0.1 to about 1 nM insulin.

4. The method according to claim 1, wherein the culture medium further comprises about 0.5 nM insulin.

5. The method according to claim 1, wherein the culture medium further comprises about 1 to about 25 mM glucose.

6. The method according to claim 1, wherein the culture medium further comprises about 5 mM glucose.

7. The method according to claim 1, wherein the population of hepatocytes and the at least one non-human, non-parenchymal cell population are maintained in vitro for at least 6 weeks.

8. The method according claim 1, wherein the human hepatocytes are primary human hepatocytes.

9. The method according to claim 1, wherein the human serum is obtained from at least one human donor suffering from a disorder of the liver.

10. The method according to claim 9, wherein the disorder of the liver is selected from Type-2 diabetes, metabolic syndrome, non-alcoholic fatty liver disease (NAFLD), non-alcoholic steatohepatitis (NASH), and cardiovascular disease.

11. The method according to claim 1, wherein at least one of the non-human, non-parenchymal cell populations comprises non-human stromal cells.

12. The method according to claim 11, wherein the stromal cells are selected from the group consisting of fibroblasts, fibroblast-derived cells, macrophages, endothelial cells, stellate cells, pericytes, inflammatory cells, cholangiocytes and other types of stromal cells, and combinations thereof.

13. The method according to claim 12, wherein the stromal cells comprise murine embryonic fibroblasts.

14. The method according to claim 13, wherein the murine embryonic fibroblasts comprise 3T3-J2 murine embryonic fibroblasts.

15. A composition comprising a population of human hepatocytes and at least one non-human, non-parenchymal cell population in co-culture in vitro, wherein the composition is incubated with a culture medium comprising human serum for at least 1 hour.

16. The composition of claim 15, wherein the culture medium comprises from about 5% to about 10% vol/vol human serum.

17. The composition of claim 15, wherein the culture medium further comprises about 0.1 to about 1 nM insulin and about 1 to about 25 mM glucose.

18. The composition of claim 15, wherein the composition is incubated with the culture medium for at least 24 hours.

19. The composition of claim 15, wherein the composition is incubated with the culture medium for at least 7 days.

20. The composition according to claim 15, wherein the population of hepatocytes and the at least one non-human, non-parenchymal cell population are maintained in vitro for at least 6 weeks.

21. A method of identifying a candidate test compound for use in treating a disorder of the liver, the method comprising:
contacting the composition according to claim 15 with the test compound;
maintaining the composition for a time and under conditions sufficient to allow an effect of the test compound on the hepatocytes;
measuring at least one indicator of hepatic function in the hepatocytes to obtain a test measurement, or applying hepatocyte imaging technology (HIAT) to the hepatocytes to obtain a test image; and
comparing the test measurement to a control measurement from the hepatocytes before contact with the test compound, or the test image to a control image of the hepatocytes before contact with the test compound,
wherein a difference between the test and control is indicative of whether the test compound is a candidate for use in treating a disorder of the liver.

22. The method according to any claim 21, wherein the at least one indicator of hepatic function is selected from the group consisting of albumin production, urea production, ATP production, glutathione production, enzyme activity, lipid accumulation, liver gene expression, and liver protein expression in the hepatocytes.

23. The method according to claim 22, wherein the enzyme activity is at least one inducible liver enzyme selected from CYP2C9 (luciferin-H), CYP3A4 (luciferin-IPA), a combination of CYP1A1, CYP1A2, CYP2B6, CPY2A6, and CYP2D6 (luciferin ME-EGE), and any combination thereof.

24. A method of determining the toxicity of a test compound, the method comprising:
contacting the composition according to claim 15 with the test compound;
maintaining the composition for a time and under conditions sufficient to allow an effect of the test compound on the hepatocytes;
measuring at least one indicator of hepatic function in the hepatocytes to obtain a test measurement, or applying hepatocyte imaging technology (HIAT) to the hepatocytes to obtain a test image; and
comparing the test measurement to a control measurement from the hepatocytes before contact with the test compound, or the test image to a control image of the hepatocytes before contact with the test compound,
wherein a difference between the test and control is indicative of hepatotoxicity of the test compound.

25. The method according to claim 24, wherein the at least one indicator of hepatic function is selected from the group consisting of albumin production, urea production, ATP production, glutathione production, enzyme activity, lipid accumulation, liver gene expression, and liver protein expression in the hepatocytes.

26. The method according to claim 25, wherein the enzyme activity is at least one inducible liver enzyme selected from CYP2C9 (luciferin-H), CYP3A4 (luciferin-IPA), a combination of CYP1A1, CYP1A2, CYP2B6, CPY2A6, and CYP2D6 (luciferin ME-EGE), and any combination thereof.

27. A method of identifying drug metabolites, the method comprising:
contacting the composition according to claim 15 with a drug;
maintaining the composition for a time and under conditions sufficient to allow the generation of metabolites; and
identifying the metabolites.

28. A method of predicting hepatic clearance of a drug, the method comprising:
contacting the composition according to claim 15 with the drug;
maintaining the composition for a time and under conditions sufficient to allow an effect of the drug on the hepatocytes;
measuring the drug concentration in the composition; and
determining a hepatic clearance value from the drug concentration measurement.
